(19) United States
(12) Patent Application Publication
(10) Pub. No.: US 2011/0009474 A1
(43) Pub. Date: Jan. 13, 2011

(54) SIRTUIN BASED METHODS AND COMPOSITIONS FOR TREATING BETA-CATENIN-RELATED CONDITIONS

(75) Inventors: David A. Sinclair, Chestnut Hill, MA (US); Ron Firestein, Brookline, MA (US); Leonard Guarente, Newton, MA (US); Gil Blander, Lexington, MA (US)

Correspondence Address: WOLF GREENFIELD & SACKS, P.C., 600 ATLANTIC AVENUE, BOSTON, MA, 02210-2206, US

(73) Assignees: President and Fellows of Harvard College, Cambridge, MA (US); Massachusetts Institute of Technology, Cambridge, MA (US)

(21) Appl. No.: 12/595,353

(22) PCT Filed: Apr. 14, 2008
(86) PCT No.: PCT/US08/60193

§ 371 Date: Sep. 16, 2010

Related U.S. Application Data
(60) Provisional application No. 60/911,448, filed on Apr. 12, 2007.

(51) Int. Cl. A61K 31/7088; A61P 35/00; A61P 17/02; A61P 13/12; A61P 19/04; A61P 17/00; A61P 37/00; C12N 5/02; G01N 33/566

(52) U.S. Cl. 514/44 R; 435/375; 436/501

(57) ABSTRACT

Provided herein are methods and compositions relating to sirtuin modulation of Wnt pathway signaling, including the use of sirtuin and sirtuin-modulating agents in the prevention and treatment of cancer and other diseases.

### BACKGROUND

The Drosophila Melanogaster Armadillo/beta-catenin protein is implicated in multiple cellular functions. The protein functions in cell signaling via the Wingless (Wg)/Wnt signaling pathway. It also functions as a cell adhesion protein at the cell membrane in a complex with E-cadherin and alpha-catenin (Cox et al. (1996) J. Cell Biol. 134: 133-148; Godt and Tepass (1998) Nature 395: 387-391; White et al. (1998) J Cell biol. 140:183-195). These two roles of beta-catenin can be separated from each other (Orsulic and Peifer (1996) J. Cell Biol. 134: 1283-1300; Sanson et al. (1996) Nature 383: 627-630).

In Wingless cell signaling, beta-catenin levels are tightly regulated by a complex containing APC, Axin, and GSK3 beta/SGG/ZW3 (Peifer et al. (1994) Development 120: 369-380).

The Wingless/beta-catenin signaling pathway is frequently mutated in human cancers, particularly those of the colon. Mutations in the tumor suppressor gene APC, as well as point mutations in beta-catenin itself lead to the stabilization of the beta-catenin protein and inappropriate activation of this pathway.

### SUMMARY

Described herein is the activation of SIRT1 as a method to modulate Wnt pathway signaling and suppress beta-catenin mediated oncogenicity.

### BRIEF DESCRIPTION OF THE DRAWINGS

FIG. 1. Generation of conditional SIRT1 transgenic mice that mimic calorie restriction induced SIRT1 overexpression. (A) Western blot analysis showing expression levels in the gut epithelium of SIRT1 in ad libitum-fed (AL) or calorie restricted (CR) rats. β-actin served as the loading control in all lanes. (B) Schematic representation of generation of single copy floxed SIRT1 transgenic mice into the Col A1 locus by FRT homing. (C), PCR confirmation of integration of the SIRT1-STOP into the Col1A locus and the removal of the STOP cassette in ES cells (D) DNA gel showing germline transmission of the Col1A-FRT locus in SIRT1 transgenic mice (E) Western blot analysis showing expression levels of SIRT1 in transgenic mice. β-actin served as the loading control in all lanes. (F) Mucin stain and immunohistochemical analysis of SIRT1 expression in the small intestine of SIRT1 transgenic animals and controls.

FIG. 2. Effect of SIRT1 overexpression on intestinal tumor formation and proliferation in Apc^{min/-} mice. (A) Pictures of whole duodenal and ileal sections show gross intestinal tumors in SIRT1 transgenic mice. Solid line indicates gastro-duodenal junction. Asterisks indicate adenomas. White bar denotes 1 mm scale. (B) Average number of tumors according to intestinal location in Apc^{min/+};SIRT1^{STOP} (n=8) and Apc^{min/-};SIRT1;Vil-Cre mice (n=11). (C) Ki-67 staining of adenomas and proliferation rates. Pictures show Ki-67 immunohistochemical staining of adenomas from Apc^{min/};SIRT1^{stop} and Apc^{min/+};SIRT1;Vil-Cre mice. Proliferation index is expressed as the percent of Ki-67 stained adenoma cells (averaged for at least 10 adenomas per cohort). Mitotic rate is calculated as the number of histologically identifiable mitotic figures per 10 high power fields (400×). Images were taken at a magnification of 400×. Values in B and C are means±s.d.

FIG. 3. SIRT1 inhibits β-catenin driven cell proliferation and transcriptional activity. (A-D). LN-CAP, DLD1, HCT116 and RKO cell lines were infected with the indicated overexpression or shRNA virus. The cells were selected and subjected to Western blot analysis with SIRT1, actin or β-catenin antibodies. The cells were seeded and cell number was monitored at the indicated time point. (E) DLD1 stable cell lines expressing Topflash-Luciferase^{PEST} were infected with the indicated constructs. Cells were subjected to western blot analysis for SIRT1 and β-catenin. The luciferase activity was normalized for total sample protein and represents three independent experiments done in quadruplicate.

FIG. 4. SIRT1 represses β-catenin transcriptional activity by directly interacting with and deacetylating β-catenin. (A) Human 293T cells were transiently transfected with HA-S33Y-β-catenin in combination with either FLAG-tagged SIRT1 or vector control. Aliquots of total protein were subjected to immunoprecipitation with anti-FLAG antibody (IP FLAG). Immunoprecipitated proteins were immunoblotted with anti-HA (upper panel) and anti-FLAG (lower panel). Left lanes contain aliquots of unprocessed extracts (input) applied directly to the gel. (B) Human 293T cells were transfected as in panel A. Proteins were immunoprecipitated with anti-HA antibody (IP HA and immunoblotted with anti-FLAG (upper panel), and anti-HA (lower panel). Left lanes contain aliquots of unprocessed extracts (input) applied directly to the gel. (C) LN-CAP cells were extracted and subjected to immunoprecipitation with anti-SIRT1 antibody or normal rabbit IgG as a control (IgG). 10% of the immunoprecipitated protein was then blotted with anti-SIRT1 (upper panel) while the remaining 90% was blotted with anti-β-catenin antibodies. (D) 293T cells were transfected with the indicated constructs and lysed 48 hr later. Comparable levels of β-catenin were immunoprecipitated using antibodies directed against the HA epitope, and Western blotted for acetylated-lysine residues (IP: HA IB: Ac-K; upper panel). The blot of the HA immunoprecipitate was reprobed using the anti-HA antibody to demonstrate approximately equal levels of the HA-β-catenin protein (IP: HA IB: HA; lower panel). (E-G) 293T cells were transfected as indicated together with the TOP-FLASH luciferase and PRL-TK Renilla luciferase reporter construct. Where indicated, nicotinamide (NAM) was added eight hours after transfection. Twenty-four hours post-transfection, the cells were harvested and subjected to luciferase assay. The data are normalized with respect to Renilla luciferase activity. The data are means±s.d. of triplicate samples.

FIG. 5 shows weight differences between SIRT1 overexpressing Apc^{min/+} mice and Apc^{min/+} control animals. Top picture illustrates degree of anemia as evidenced by paw color.

FIG. 6 shows targeting of SIRT1-STOP plasmid to ColA1 locus using flp recombinase technology. (A) DNA gel illustrating integration of SIRT1-STOP into the Col1A locus by PCR (B), removal of the STOP cassette in ES cells (C) and SIRT1 expression in transgenic mice in which the STOP cassette has been deleted by breeding with a Cre animal (D).

FIG. 7 shows the nucleotide and amino acid sequences of human SIRT1 and human β-catenin (SEQ ID NOs: 1-4).

FIG. 8 is a series of photographic images of cell colonies of SIRT transfected cells, demonstrating that overexpression of wild-type SIRT1 reduces colony formation in soft agar while overexpression of a dominant-negative SIRT1 has no effect on colony formation.

FIG. 9 is a bar graph quantitating the reduction in foci formation in cells overexpressing SIRT1, as measured in foci per 50 high power fields (“HPF”).

### DETAILED DESCRIPTION

The subject matter described herein is based at least in part on the discovery that SIRT1 deacetylates β-catenin.

### Definitions

For convenience, certain terms employed in the specification, examples, and appended claims are collected here. Unless defined otherwise, all technical and scientific terms used herein have the same meaning as commonly understood by one of ordinary skill in the art to which this invention belongs.

The articles “a” and “an” are used herein to refer to one or to more than one (i.e., to at least one) of the grammatical object of the article. By way of example, “an element” means one element or more than one element.

The term “acetylase” is used interchangeable herein with “acetyl transferase” and refers to an enzyme that catalyzes the addition of an acetyl group (CH₃CO⁻) to an amino acid. Exemplary acetyl transferases, such as histone acetyl transferases (HAT), include but are not limited to CREB-binding protein (CBP), p300/CBP-associated factor (PCAF); general control non-repressed 5 (GCN5); TBP-associated factor (TAF250); steroid receptor coactivator (SCR1) and monocytic leukemia zinc finger protein (MOZ).

The term “agent” is used herein to denote a chemical compound, a mixture of chemical compounds, a biological macromolecule (such as a nucleic acid, an antibody, a protein or portion thereof, e.g., a peptide), or an extract made from biological materials such as bacteria, plants, fungi, or animal (particularly mammalian) cells or tissues. Agents may be identified as having a particular activity by screening assays described herein below. The activity of such agents may render it suitable as a “therapeutic agent” which is a biologically, physiologically, or pharmacologically active substance (or substances) that acts locally or systemically in a subject.

The term “interact” or “interaction” as used herein is meant to include detectable relationships or association (e.g. biochemical interactions) between molecules, such as interaction between protein-protein, protein-nucleic acid, nucleic acid-nucleic acid, and protein-small molecule or nucleic acid-small molecule in nature.

A composition may be a pharmaceutical composition, comprising, e.g., a pharmaceutically acceptable buffer or vehicle, such as further described herein. A composition may comprise additional molecules necessary for an acetylation or deacetylation reaction.

The term “isolated,” when used in the context of a protein, polypeptide or peptide, refers to polypeptides, peptides or proteins that are isolated from other cellular proteins and is meant to encompass both purified and recombinant polypeptides.

A “naturally occurring compound” refers to a compound that can be found in nature, i.e., a compound that has not been designed by man. A naturally occurring compound may have been made by man or by nature.

The term “percent identical” refers to sequence identity between two amino acid sequences or between two nucleotide sequences. Identity can each be determined by comparing a position in each sequence which may be aligned for purposes of comparison. When an equivalent position in the compared sequences is occupied by the same base or amino acid, then the molecules are identical at that position; when the equivalent site occupied by the same or a similar amino acid residue (e.g., similar in steric and/or electronic nature), then the molecules can be referred to as homologous (similar) at that position. Expression as a percentage of homology, similarity, or identity refers to a function of the number of identical or similar amino acids at positions shared by the compared sequences. Expression as a percentage of homology, similarity, or identity refers to a function of the number of identical or similar amino acids at positions shared by the compared sequences. Various alignment algorithms and/or programs may be used, including FASTA, BLAST, or ENTREZ. FASTA and BLAST are available as a part of the GCG sequence analysis package (University of Wisconsin, Madison, Wis.), and can be used with, e.g., default settings. ENTREZ is available through the National Center for Biotechnology Information, National Library of Medicine, National Institutes of Health, Bethesda, Md. In one embodiment, the percent identity of two sequences can be determined by the GCG program with a gap weight of 1, e.g., each amino acid gap is weighted as if it were a single amino acid or nucleotide mismatch between the two sequences.

Other techniques for alignment are described in Methods in Enzymology, vol. 266: Computer Methods for Macromolecular Sequence Analysis (1996), ed. Doolittle, Academic Press, Inc., a division of Harcourt Brace & Co., San Diego, Calif., USA. Preferably, an alignment program that permits gaps in the sequence is utilized to align the sequences. The Smith-Waterman is one type of algorithm that permits gaps in sequence alignments. See Meth. Mol. Biol. 70: 173-187 (1997). Also, the GAP program using the Needleman and Wunsch alignment method can be utilized to align sequences. An alternative search strategy uses MPSRCH software, which runs on a MASPAR computer. MPSRCH uses a Smith-Waterman algorithm to score sequences on a massively parallel computer. This approach improves ability to pick up distantly related matches, and is especially tolerant of small gaps and nucleotide sequence errors. Nucleic acid-encoded amino acid sequences can be used to search both protein and DNA databases.

The terms “polynucleotide”, and “nucleic acid” are used interchangeably. They refer to a polymeric form of nucleotides of any length, either deoxyribonucleotides or ribonucleotides, or analogs thereof Polynucleotides may have any three-dimensional structure, and may perform any function, known or unknown. The following are non-limiting examples of polynucleotides: coding or non-coding regions of a gene or gene homolog, loci (locus) defined from linkage analysis, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified, such as by conjugation with a labeling component. The term “recombinant” polynucleotide means a polynucleotide of genomic, cDNA, semisynthetic, or synthetic origin which either does not occur in nature or is linked to another polynucleotide in a nonnatural arrangement.

The term “small molecule” is art-recognized and refers to a composition which has a molecular weight of less than about 2000 amu, or less than about 1000 amu, and even less than about 500 amu. Small molecules may be, for example, nucleic acids, peptides, polypeptides, peptide nucleic acids, peptidomimetics, carbohydrates, lipids or other organic (carbon containing) or inorganic molecules. Many pharmaceutical companies have extensive libraries of chemical and/or biological mixtures, often fungal, bacterial, or algal extracts, which can be screened with any of the assays described herein. The term “small organic molecule” refers to a small molecule that is often identified as being an organic or medicinal compound, and does not include molecules that are exclusively nucleic acids, peptides or polypeptides.

The term “substantially homologous” when used in connection with amino acid sequences, refers to sequences which are substantially identical to or similar in sequence with each other, giving rise to a homology of conformation and thus to retention, to a useful degree, of one or more biological (including immunological) activities. The term is not intended to imply a common evolution of the sequences.

“Substantially purified” refers to a protein that has been separated from components which naturally accompany it. Preferably the protein is at least about 80%, more preferably at least about 90%, and most preferably at least about 99% of the total material (by volume, by wet or dry weight, or by mole percent or mole fraction) in a sample. Purity can be measured by any appropriate method, e.g., in the case of polypeptides by column chromatography, gel electrophoresis or HPLC analysis.

“Transcriptional regulatory sequence” is a generic term used throughout the specification to refer to DNA sequences, such as initiation signals, enhancers, and promoters, which induce or control transcription of protein coding sequences with which they are operable linked. In preferred embodiments, transcription of one of the recombinant genes is under the control of a promoter sequence (or other transcriptional regulatory sequence) which controls the expression of the recombinant gene in a cell-type which expression is intended. It will also be understood that the recombinant gene can be under the control of transcriptional regulatory sequences which are the same or which are different from those sequences which control transcription of the naturally-occurring forms of genes as described herein.

The term “treating” a condition or disease is art-recognized and refers to curing as well as ameliorating at least one symptom of a condition or disease or preventing the condition or disease from worsening.

A “vector” is a self-replicating nucleic acid molecule that transfers an inserted nucleic acid molecule into and/or between host cells. The term includes vectors that function primarily for insertion of a nucleic acid molecule into a cell, replication of vectors that function primarily for the replication of nucleic acid, and expression vectors that function for transcription and/or translation of the DNA or RNA. Also included are vectors that provide more than one of the above functions. As used herein, “expression vectors” are defined as polynucleotides which, when introduced into an appropriate host cell, can be transcribed and translated into a polypeptide(s). An “expression system” usually connotes a suitable host cell comprised of an expression vector that can function to yield a desired expression product.

The term “therapeutic agent” is art-recognized and refers to any chemical moiety that is a biologically, physiologically, or pharmacologically active substance that acts locally or systemically in a subject. Examples of therapeutic agents, also referred to as “drugs”, are described in well-known literature references such as the Merck Index, the Physicians Desk Reference, and The Pharmacological Basis of Therapeutics, and they include, without limitation, medicaments; vitamins; mineral supplements; substances used for the treatment, prevention, diagnosis, cure or mitigation of a disease or illness; substances which affect the structure or function of the body; or pro-drugs, which become biologically active or more active after they have been placed in a physiological environment.

The term “therapeutic effect” is art-recognized and refers to a local or systemic effect in animals, particularly mammals, and more particularly humans caused by a pharmacologically active substance. The term thus means any substance intended for use in the diagnosis, cure, mitigation, treatment or prevention of disease or in the enhancement of desirable physical or mental development and/or conditions in an animal or human. The phrase “therapeutically-effective amount” means that amount of such a substance that produces some desired local or systemic effect at a reasonable benefit/risk ratio applicable to any treatment. The therapeutically effective amount of such substance will vary depending upon the subject and disease condition being treated, the weight and age of the subject, the severity of the disease condition, the manner of administration and the like, which can readily be determined by one of ordinary skill in the art. For example, certain compositions described herein may be administered in a sufficient amount to produce a at a reasonable benefit/risk ratio applicable to such treatment.

When using the term “comprising” herein, it will be understood that in certain embodiments, the term can be substituted for “consisting of or “consisting essentially of.”

The term “small molecule” is art-recognized and refers to a composition which has a molecular weight of less than about 2000 amu, or less than about 1000 amu, and even less than about 500 amu. Small molecules may be, for example, nucleic acids, peptides, polypeptides, peptide nucleic acids, peptidomimetics, carbohydrates, lipids or other organic (carbon containing) or inorganic molecules. Many pharmaceutical companies have extensive libraries of chemical and/or biological mixtures, often fungal, bacterial, or algal extracts, which can be screened with any of the assays described herein. The term “small organic molecule” refers to a small molecule that is often identified as being an organic or medicinal compound, and does not include molecules that are exclusively nucleic acids, peptides or polypeptides.

The term “prophylactic” or “therapeutic” treatment is art-recognized and refers to administration of a drug to a host. If it is administered prior to clinical manifestation of the unwanted condition (e.g., disease or other unwanted state of the host animal) then the treatment is prophylactic, i.e., it protects the host against developing the unwanted condition, whereas if administered after manifestation of the unwanted condition, the treatment is therapeutic (i.e., it is intended to diminish, ameliorate or maintain the existing unwanted condition or side effects therefrom).

A “patient,” “subject” or “host” to be treated by the subject method may mean either a human or non-human animal.

The term “mammal” is known in the art, and exemplary mammals include humans, primates, bovines, porcines, canines, felines, and rodents (e.g., mice and rats).

The term “bioavailable” when referring to a compound is art-recognized and refers to a form of a compound that allows for it, or a portion of the amount of compound administered, to be absorbed by, incorporated to, or otherwise physiologically available to a subject or patient to whom it is administered.

The term “pharmaceutically-acceptable salts” is art-recognized and refers to the relatively non-toxic, inorganic and organic acid addition salts of compounds, including, for example, those contained in compositions described herein.

The term “pharmaceutically acceptable carrier” is art-recognized and refers to a pharmaceutically-acceptable material, composition or vehicle, such as a liquid or solid filler, diluent, excipient, solvent or encapsulating material, involved in carrying or transporting any subject composition or component thereof from one organ, or portion of the body, to another organ, or portion of the body. Each carrier must be “acceptable” in the sense of being compatible with the subject composition and its components and not injurious to the patient. Some examples of materials which may serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The terms “systemic administration,” “administered systemically,” “peripheral administration” and “administered peripherally” are art-recognized and refer to the administration of a subject composition, therapeutic or other material other than directly into the central nervous system, such that it enters the patient's system and, thus, is subject to metabolism and other like processes.

The terms “parenteral administration” and “administered parenterally” are art-recognized and refer to modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intra-articulare, subcapsular, subarachnoid, intraspinal, and intrasternal injection and infusion.

### Exemplary Compositions

Provided herein are compositions comprising a sirtuin protein or a homolog thereof and a β-catenin protein or homolog thereof. Also described herein are protein complexes, e.g., isolated protein complexes, such as a protein complex comprising a sirtuin protein or a homolog thereof and a β-catenin protein or homolog thereof.

The proteins and other compositions of matter described herein may be from a eukaryote or a prokaryote, from a single cell, single cell organism or from a multicellular organism. The compositions of matter may be mammalian, vertebrate, yeast, human or non-human. For example, a sirtuin protein and a β-catenin protein may be from a human.

A sirtuin may be SIRT1. A sirtuin may also be another member of the family of sirtuins, e.g., SIRT2, 3, 4, 5, 6 or 7. “Sirtuin deacetylase protein family members;” “Sir2 family members;” “Sir2 protein family members” and “sirtuin proteins” are used interchangeably herein and includes yeast Sir2, Sir-2.1, and human SIRT1-7. The mouse homolog of human SIRT1 is Sirt2α. Other family members include the four additional yeast Sir2-like genes termed “HST genes” (homologues of Sir two) HST1, HST2, HST3 and HST4 (Brachmann et al. (1995) Genes Dev. 9:2888 and Frye et al. (1999) BBRC 260:273). A subgroup of sirtuins are those that share more similarities with human SIRT1 and/or yeast Sir2 than with human SIRT2, such as those members having at least part of the N-terminal sequence present in SIRT1 and absent in SIRT2, such as SIRT3 has.

Nucleotide and amino acid sequences of human sirtuins and exemplary conserved domains are set forth below:

| Sirt | | nucleotide | amino acid | conserved |
|---|---|---|---|---|
| domains | | sequence | sequence | (amino acids) |
| SIRT1 | | NM_012238 | NP_036370 | 431-536; 254-489 |
| SIRT2 | i1 | NM_012237 | NP_036369 | 77-331 |
| | i2 | NM_030593 | NP_085096 | 40-294 |
| SIRT3 | ia | NM_012239 | NP_036371 | 138-373 |
| | ib | NM_001017524 | NP_001017524 | 1-231 |
| SIRT4 | | NM_012240 | NP_036372 | 47-308 |
| SIRT5 | i1 | NM_012241 | NP_036373 | 51-301 |
| | i2 | NM_031244 | NP_112534 | 51-287 |
| SIRT6 | | NM_016539 | NP_057623 | 45-257 |
| SIRT7 | | NM_016538 | NP_057622 | 100-314 |

The nucleotide and amino acid sequences of the human sirtuin, SIRT1 (silent mating type information regulation 2 homolog), are set forth as SEQ ID NOs: 1 and 2, respectively (corresponding to GenBank Accession numbers NM_{—}012238 and NP_{—}036370, respectively).

Human β-catenin nucleotide and amino acid sequences are provided in GenBank Accession numbers NM_{—}001904.2→NP_{—}001895.1, respectively (SEQ ID NOs: 3 and 4). Conserved domains include about amino acids 399-518; 229-348; 483-623; 108-222 and 350-390.

A homolog of a protein or reference protein, e.g., a sirtuin protein or a β-catenin protein, refers to a protein that differs from the reference protein but that can be used for the same purpose as the reference protein. For example, a homolog of a reference protein may be a homolog of the reference protein or a protein having a certain amino acid sequence homology to that of the full length reference protein or to that of a homolog of the reference protein.

A homolog of a protein may be a biologically active homolog. A biologically active homolog of a sirtuin may be a homolog that is capable of (or sufficient for) binding to a β-catenin protein or a homolog that is capable of deacetylating a β-catenin protein. A biologically active homolog of a β-catenin protein may be a homolog that is capable of (or sufficient for) binding to a sirtuin or a homolog that comprises the amino acid region that is acetylated.

Binding between two proteins may be significant binding, e.g., binding with an affinity that is higher than the binding affinity between one of the proteins and another unrelated protein. For example, a binding affinity between two proteins may be at least about 10⁻⁶, 10⁻⁷, 10⁻⁸, 10⁻⁹ or 10⁻¹⁰ M.

A homolog of a protein may be at least about 10, 20, 50, 75, 100, 150, 200, 250, 300 or more (consecutive) amino acids long. A homolog may comprise fewer amino acids than the full length naturally occurring protein. For example, a homolog of a protein may be a protein or peptide that is lacking about 1, 2, 3, 4, 5, 10, 25, 50, 75, 100, 150, 175, 200 or more contiguous amino acids at the N- and/or C-terminus of the protein relative to the full length protein, e.g., the naturally occurring full length protein.

A homolog of a protein or a full length protein may comprise one or more heterologous or unrelated amino acids at the N- and/or C-terminus. For example, a homolog of or a full length sirtuin or a β-catenin protein may be linked to about 1, 2, 3, 4, 5, 10, 25, 50, 75, 100, 150, 175, 200 or more contiguous amino acids at the N- or C-terminus of the protein, which amino acids form an amino acid sequence that is unrelated to a sequence that is present in the full length sirtuin or β-catenin protein, respectively, at least not in the same position. An amino acid sequence that is unrelated may be referred to as being heterologous.

A homolog of a protein, e.g., a homolog of a sirtuin protein or of a β-catenin protein, may be a biologically active homolog. A biologically active homolog of a sirtuin may be a homolog that is capable of (or sufficient for) binding to a β-catenin protein or a homolog that is capable of deacetylating a β-catenin protein. A biologically active homolog of a β-catenin protein may be a homolog that is capable of (or sufficient for) binding to a sirtuin or a homolog that comprises the amino acid region that is acetylated.

A biologically active homolog of a sirtuin may comprise its active site or catalytic site that is involved in deacetylation, e.g. its core domain. For example, a biologically active homolog of a sirtuin comprises all of or a portion of the conserved domain listed in the Table above. Biologically active portions of sirtuins may comprise the core domain of sirtuins. For example, amino acids 62-293 of SIRT1 having SEQ ID NO: 2, which are encoded by nucleotides 237 to 932 of SEQ ID NO: 1, encompass the NAD⁺ binding domain and the substrate binding domain. Therefore, this region is sometimes referred to as the core domain. Other biologically active portions of SIRT1, also sometimes referred to as core domains, include about amino acids 261 to 447 of SEQ ID NO: 2, which are encoded by nucleotides 834 to 1394 of SEQ ID NO: 1; about amino acids 242 to 493 of SEQ ID NO: 2, which are encoded by nucleotides 777 to 1532 of SEQ ID NO: 1; or about amino acids 254 to 495 of SEQ ID NO: 2, which are encoded by nucleotides 813 to 1538 of SEQ ID NO: 1.

A biologically active homolog of a β-catenin protein may comprise the site that is acetylated and which is deacetylated by SIRT1. Known acetylated residues of human β-catenin are K49 and K345. Accordingly, a biologically active homolog of a β-catenin protein may be a protein or peptide encompassing one or both of these residues, e.g., one having a certain number of amino acids as further described herein.

A homolog of a reference protein may be a protein comprising an amino acid sequence that is at least about 70%, 80%, 90%, 95%, 98% or 99% identical to that of the reference protein. For example, a homolog of a sirtuin protein or homolog thereof may be a protein that comprises an amino acid sequence that is at least about 70%, 80%, 90%, 95%, 98% or 99% identical to that of the sirtuin or homolog thereof. A homolog of a β-catenin protein or homolog thereof may be a protein that comprises an amino acid sequence that is at least about 70%, 80%, 90%, 95%, 98% or 99% identical to that of the β-catenin or homolog thereof.

Amino acid sequences of proteins may differ, e.g., from SEQ ID NO: 2 or 4 in the addition, deletion, or substitution of 1, 2, 3, 5, 10, 15 or 20 amino acids. Amino acid substitutions may be with conserved amino acids. Conservative substitutions may be defined herein as exchanges within one of the following five groups: I. Small aliphatic, nonpolar or slightly polar residues: Ala, Ser, Thr, Pro, Gly; II. Polar, negatively charged residues and their amides: Asp, Asn, Glu, Gln; III. Polar, positively charged residues: His, Arg., Lys; IV. Large, aliphatic nonpolar residues: Met, Leu, Ile, Val, Cys; and V. Large aromatic residues: Phe, Try, Trp. Within the foregoing groups the following five substitutions are considered “highly conservative”: Asp/Glu; His/Arg/Lys; Phe/Tyr/Trp; Met/Leu/Ile/Val. Semi-conservative substitutions are defined to be exchanges between two of groups (I)-(V) above which are limited to supergroup (A), comprising (I), (II), and (III) above, or to supergroup (B), comprising (IV) and (V) above. Amino acid deletions, additions or substitutions are preferably located in areas of a protein that is not required for biological activity, e.g., those further described herein.

A homolog of a reference protein or homolog thereof may also be a protein that is encoded by a nucleic acid consisting of a nucleotide sequence that is at least about 70%, 80%, 90%, 95%, 98% or 99% identical to that of a nucleic acid encoding the reference protein or a homolog thereof.

A homolog of a reference protein or homolog thereof may also be a protein that is encoded by a nucleic acid that hybridizes to a nucleic acid that encodes the reference protein or the homolog thereof. Hybridization can be conducted under low or high stringency conditions. Appropriate stringency conditions which promote DNA hybridization, for example, 6.0× sodium chloride/sodium citrate (SSC) at about 45° C., followed by a wash of 2.0×SSC at 50° C., are known to those skilled in the art or can be found in Current Protocols in Molecular Biology, John Wiley & Sons, N.Y. (1989), 6.3.1-6.3.6. For example, the salt concentration in the wash step can be selected from a low stringency of about 2.0×SSC to a high stringency of about 0.2×SSC. In addition, the temperature in the wash step can be increased from low stringency conditions at room temperature, about 22° C., to high stringency conditions at about 65° C. Both temperature and salt may be varied, or temperature of salt concentration may be held constant while the other variable is changed. Preferred nucleic acids are those that hybridize to a nucleic acid comprising SEQ ID NO: 1 or 3 or a portion thereof under high stringency conditions, such as hybridization and wash conditions in 0.2×SSC at 65° C.

Also provided are compositions comprising one or more nucleic acids encoding a sirtuin protein or homolog thereof and a β-catenin protein or homolog thereof. In one embodiment, a nucleic acid encodes a sirtuin protein or a homolog thereof and a β-catenin protein or homolog thereof. A composition may also comprise one nucleic acid encoding a sirtuin protein or homolog thereof and one nucleic acid encoding a β-catenin protein or a homolog thereof.

A protein may be linked directly or indirectly to one or more amino acids, e.g., to a heterologous amino acid sequence or peptide and may form a fusion protein. Heterologous amino acid sequences may provide stability, solubility or merely mark a protein for detection and/or isolation. For example, protein may be fused or linked to a histidine tag or to a portion of an immunoglobulin molecule, such as a hinge, CH2 and/or CH3 domain.

A nucleic acid encoding a protein, e.g., a sirtuin or a β-catenin or a homolog thereof, may further be linked to one or more regulatory elements, e.g., a promoter. A nucleic acid may be part of a plasmid or a vector, e.g., an expression vector. Exemplary expression vectors include viral or non-viral vectors, such as adenovirus vectors, adeno-associated virus vectors, retrovirus vectors, lentivirus vectors, and plasmid vectors. Exemplary types of viruses include HSV (herpes simplex virus), AAV (adeno associated virus), HIV (human immunodeficiency virus), BIV (bovine immunodeficiency virus), and MLV (murine leukemia virus). Nucleic acids can be administered in any desired format that provides sufficiently efficient delivery levels, including in virus particles, in liposomes, in nanoparticles, and complexed to polymers.

One or more nucleic acids may be comprised in a cell, e.g., an isolated cell or a cell within an organism. Nucleic acids may also be present in one or more cells of an animal and thereby form, e.g., a transgenic animal. Exemplary transgenic animals are non-human transgenic animals comprising a tissue specific and conditional SIRT1, e.g., as further described in the Examples.

Further provided are antibodies and homologs thereof that bind specifically to a complex comprising a sirtuin protein and a β-catenin protein. The antibodies preferably do not bind specifically to a sirtuin alone or to a β-catenin alone, such that the antibodies may be used to specifically detect a complex between a sirtuin and a β-catenin protein. Antibodies may bind with more affinity to the complex than to one or the two proteins separately. Antibodies may be polyclonal or monoclonal antibodies and may be an IgG, IgD, IgM, IgA, or IgE antibody. Antibodies may be humanized, chimeric, single chain, or human.

### Therapeutic Methods

Provided herein are methods for treating or preventing a disease or condition that may benefit from reducing β-catenin activity or levels, e.g., a disease that is associated with a dysregulated activation of β-catenin activity. A method may comprise administering to a subject in need thereof a therapeutically effective amount of an agent that increases the level or activity of a sirtuin, e.g., SIRT1.

In one embodiment, an agent that increases sirtuin level or activity is a small molecule that increases the activity of a sirtuin. “Activating a sirtuin protein” refers to the action of producing an activated sirtuin protein, i.e., a sirtuin protein that is capable of performing at least one of its biological activities to at least some extent, e.g., with an increase of activity of at least about 10%, 50%, 2 fold or more. Biological activities of sirtuin proteins include deacetylation, e.g., of histones, p53 and β-catenin; extending lifespan; increasing genomic stability; silencing transcription; and controlling the segregation of oxidized proteins between mother and daughter cells. A “sirtuin activating compound” refers to a compound that activates a sirtuin protein, stimulates or increases at least one of its activities, or increases the level of a sirtuin protein, or a combination thereof. In an exemplary embodiment, a sirtuin -activating compound may increase at least one biological activity of a sirtuin protein by at least about 1%, 5%, 10%, 25%, 50%, 75%, 100%, or more. Exemplary biological activities of sirtuin proteins include deacetylation, e.g., of histones and p53; extending lifespan; increasing genomic stability; silencing transcription; and controlling the segregation of oxidized proteins between mother and daughter cells. A “sirtuin-inhibiting compound” refers to a compound that decreases the level of a sirtuin protein and/or decreases at least one activity of a sirtuin protein. In an exemplary embodiment, a sirtuin-inhibiting compound may decrease at least one biological activity of a sirtuin protein by at least about 1%, 5%, 10%, 25%, 50%, 75%, 100%, or more. Exemplary biological activities of sirtuin proteins include deacetylation, e.g., of histones and p53; extending lifespan; increasing genomic stability; silencing transcription; and controlling the segregation of oxidized proteins between mother and daughter cells. A “sirtuin-modulating compound” refers to a compound as described herein. In exemplary embodiments, a sirtuin-modulating compound may either up regulate (e.g., activate or stimulate), down regulate (e.g., inhibit or suppress) or otherwise change a functional property or biological activity of a sirtuin protein. Sirtuin-modulating compounds may act to modulate a sirtuin protein either directly or indirectly. In certain embodiments, a sirtuin-modulating compound may be a sirtuin-activating compound or a sirtuin-inhibiting compound.

Diseases or conditions that may benefit from a sirtuin activator include those that are associated with a dysregulated activation of β-catenin activity. At least in part because elevated β-catenin activity is associated with proliferating or hyper-proliferating cells, a condition that is associated with, or characterized by the presence of, hyper-proliferating cells can be treated or prevented as described herein.

Uncontrolled activation of β-catenin has been implicated in 90% of colorectal cancers as well as other cancers such as melanoma, glioblastoma, prostate and breast. Downregulation of beta-catenin activity leads to cancer cell death and tumor regression in animal models suggesting the protein is an important target for cancer therapy. Accordingly, exemplary diseases or states that may be treated include cancer, including benign, malignant or metastatic cancer. Particular examples of cancer are age-related cancer, e.g., cancer of the colon, lung, skin (e.g., melanoma), liver (hepatocellular carcinoma and hepatoblastoma) and ovary. Other cancers include prostate cancer, breast cancer, meduloblastoma, philomatricoma and glioblastoma. Methods described herein may reduce the number and/or size of tumors. In the case of colon cancer, methods described herein may reduce the number and/or size of adenomas, e.g., in the intestinal tract, such as within the small intestine and/or colon. The methods may also reduce tumor morbidity, such as colon tumor morbidity in colon cancer. Generally, small molecule modulators of SIRT1 may be used in cancer chemotherapy, cancer chemoprevention, and as adjunct therapies to existing treatments for cancer.

An agent that increase the level or activity of a sirtuin may be contacted with a cell that is hyper-proliferating. For example, an agent may be contacted with the intestinal tract of a subject having intestinal, e.g., colon, polyps or tumors. To achieve this local delivery, an agent may be administered orally in a form in which it will be delivered to the intestinal tract or the colon of the subject to whom it is administered.

Where the agent is a heterologous nucleic acid encoding SIRT1 or a biologically active homolog thereof, the nucleic acid may be targeted to and expressed in the intestinal tract of the subject.

Based at least in part on the fact that β-catenin regulates the signaling of E-cadherin, uses of the compositions and methods described herein include treating cancer, diseases of airway obstruction (such as asthma and chronic obstructive pulmonary disease (COPD)), polycystic kidney disease (ADPKD); Hailey-Hailey disease; Sjogren's disease with SIRT1 modulators. Other diseases that may be treated or prevented as described herein include wounds (wound healing), fibromatosis, osteoporosis, ischemic neuronal death and endometriosis.

Also provided herein are methods for treating a disease, condition or state that can benefit from increasing the activity of β-catenin in a subject afflicted therewith. A method may comprise administering to a subject in need thereof a therapeutically effective amount of an agent that decreases the level or activity of a sirtuin. An agent may be a compound that inhibits the activity of a sirtuin, e.g., SIRT1. “Inhibiting a sirtuin protein” refers to the action of reducing at least one of the biological activities of a sirtuin protein to at least some extent, e.g., at least about 10%, 50%, 2 fold or more. Exemplary diseases include those in which it is desirable to stimulate cell proliferation.

Exemplary sirtuin modulators, including activators and inhibitors, are described, e.g., in U.S. patent applications having publication numbers 20050096256; 20050136537; 20050171027; 20050267023; 20060025337; 20060084085; 20060111435; 20060229265; 20060276416; 20060276393; 20070014833; 20070037809; 20070037827; 20070037865; 20070043050; 20070015809; 20070037810; 20070077652; 20070099830; 20070105109; 20070117765; 20070149466; 20070149495; 20070160586; 20070173527; 20070185049; 20070197459; 20070212395; 20070225246; 20070248590; 20080015247; 20080021063; 20080032987; 20080045610; and 20080070991, and PCT applications having publication numbers WO2007019344; WO2007008548; WO2006127987; WO2006105440; WO2006094248; WO2006094246; WO2006094239; WO2006094237; WO2006094236; WO2006094235; WO2006094233; WO2006094210; WO2006094209; WO2006079021; WO2006078941; WO2006076681; WO2007005453; WO2006138418; WO2006096780; WO2006068656. All the activators and inhibitors of sirtuins that are described in these publications are specifically incorporated by reference herein. Exemplary activators and inhibitors are also set forth in Exhibits A, B and C, attached hereto. The compounds set forth in Exhibits A and B are specifically incorporated by reference herein.

Also included are pharmaceutically acceptable addition salts and complexes of the sirtuin activator and inhibiting compounds. In cases wherein the compounds may have one or more chiral centers, unless specified, the compounds contemplated herein may be a single stereoisomer or racemic mixtures of stereoisomers.

In cases in which the compounds have unsaturated carbon-carbon double bonds, both the cis (Z) and trans (E) isomers are contemplated herein. In cases wherein the compounds may exist in tautomeric forms, such as keto-enol tautomers, such as

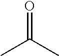

and

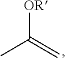

each tautomeric form is contemplated as being included within the methods presented herein, whether existing in equilibrium or locked in one form by appropriate substitution with R′. The meaning of any substituent at any one occurrence is independent of its meaning, or any other substituent's meaning, at any other occurrence.

Also included in the methods presented herein are prodrugs of the compounds. Prodrugs are considered to be any covalently bonded carriers that release the active parent drug in vivo. Metabolites, such as in vivo degradation products, of the compounds described herein are also included.

Analogs and derivatives of the above-described compounds can also be used for activating a member of the sirtuin protein family. For example, derivatives or analogs may make the compounds more stable or improve their ability to traverse cell membranes or being phagocytosed or pinocytosed. Exemplary derivatives include glycosylated derivatives, as described, e.g., in U.S. Pat. No. 6,361,815 for resveratrol. Other derivatives of resveratrol include cis- and trans-resveratrol and conjugates thereof with a saccharide, such as to form a glucoside (see, e.g., U.S. Pat. No. 6,414,037). Glucoside polydatin, referred to as piceid or resveratrol 3-O-beta-D-glucopyranoside, can also be used. Saccharides to which compounds may be conjugated include glucose, galactose, maltose, lactose and sucrose. Glycosylated stilbenes are further described in Regev-Shoshani et al. Biochemical J. (published on Apr. 16, 2003 as BJ20030141). Other derivatives of compounds described herein are esters, amides and prodrugs. Esters of resveratrol are described, e.g., in U.S. Pat. No. 6,572,882. Resveratrol and derivatives thereof can be prepared as described in the art, e.g., in U.S. Pat. Nos. 6,414,037; 6,361,815; 6,270,780; 6,572,882; and Brandolini et al. (2002) J. Agric. Food. Chem. 50:7407. Derivatives of hydroxyflavones are described, e.g., in U.S. Pat. No. 4,591,600. Resveratrol and other activating compounds can also be obtained commercially, e.g., from Sigma.

Agents may be naturally-occurring or non-naturally occurring. If naturally-occurring, they may be isolated from their normal environment. For example, a composition comprising an agent that modulates sirtuin activity may comprise at least about 80%, 90%, 95%, 98% or 99% (e.g., by weight) of the agent relative to other components, such as relative to other molecules or other proteins. An agent may be isolated or purified from its natural environment. Accordingly, if an activating compound occurs naturally, it may be at least partially isolated from its natural environment prior to use. For example, a plant polyphenol may be isolated from a plant and partially or significantly purified prior to use in the methods described herein. An activating compound may also be prepared synthetically, in which case it would be free of other compounds with which it is naturally associated. In an illustrative embodiment, an activating composition comprises, or an activating compound is associated with, less than about 50%, 10%, 1%, 0.1%, 10⁻²% or 10⁻³% of a compound with which it is naturally associated.

A cell may be contacted with a solution having a concentration of an activating or inhibiting compound of less than about 0.1 μM; 0.5 μM; less than about 1 μM; less than about 10 μM or less than about 100 μM; more than about 1, 10, 100, or 500 μM; or more than about 1 mM, 10 mM or 100 mM. The concentration of the activating compound may also be in the range of about 0.1 to 1 μM, about 1 to 10 μM, about 10 to 100 μM, about 100 μM to 1 mM or about 1 mM to 100 mM. The appropriate concentration may depend on the particular compound and the particular cell used as well as the desired effect. For example, a cell may be contacted with a “sirtuin activating” or a “sirtuin inhibitory” concentration of an activating or inhibiting compound, respectively, e.g., a concentration sufficient for activating or inhibiting the sirtuin by a factor of at least 10%, 30%, 50%, 100%, 3, 10, 30, or 100 fold, respectively.

In certain embodiments, methods comprise using an agent that increases the activity of a sirtuin, with the proviso that the agent is not a particular molecule, such as a molecule described herein. For example, in certain methods, the agent is not resveratrol; in certain methods, the agent is not resveratrol or a derivative, e.g., a metabolite, thereof; in certain methods the agent is not a flavone, a stilbene, or a chalcone; and in certain embodiments, the agent is not naturally-occurring.

In certain embodiments, the subject sirtuin activators, such as SIRT1 activators, do not have any substantial ability to inhibit PI3-kinase, inhibit aldoreductase and/or inhibit tyrosine protein kinases at concentrations (e.g., in vivo) effective for activating the deacetylase activity of the sirtuin, e.g., SIRT1. For instance, in preferred embodiments the sirtuin activator is chosen to have an EC₅₀ for activating sirtuin deacetylase activity that is at least 5 fold less than the EC₅₀ for inhibition of one or more of aldoreductase and/or tyrosine protein kinases, and even more preferably at least 10 fold, 100 fold or even 1000 fold less.

In certain embodiments, the subject sirtuin activators do not have any substantial ability to transactivate EGFR tyrosine kinase activity at concentrations (e.g., in vivo) effective for activating the deacetylase activity of the sirtuin. For instance, in preferred embodiments the sirtuin activator is chosen to have an EC₅₀ for activating sirtuin deacetylase activity that is at least 5 fold less than the EC₅₀ for transactivating EGFR tyrosine kinase activity, and even more preferably at least 10 fold, 100 fold or even 1000 fold less.

In certain embodiments, the subject sirtuin activators do not have any substantial ability to cause coronary dilation at concentrations (e.g., in vivo) effective for activating the deacetylase activity of the sirtuin. For instance, in preferred embodiments the sirtuin activator is chosen to have an EC₅₀ for activating sirtuin deacetylase activity that is at least 5 fold less than the EC₅₀ for coronary dilation, and even more preferably at least 10 fold, 100 fold or even 1000 fold less.

In certain embodiments, the subject sirtuin activators do not have any substantial spasmolytic activity at concentrations (e.g., in vivo) effective for activating the deacetylase activity of the sirtuin. For instance, in preferred embodiments the sirtuin activator is chosen to have an EC₅₀ for activating sirtuin deacetylase activity that is at least 5 fold less than the EC₅₀ for spasmolytic effects (such as on gastrointestinal muscle), and even more preferably at least 10 fold, 100 fold or even 1000 fold less.

In certain embodiments, the subject sirtuin activators do not have any substantial ability to inhibit hepatic cytochrome P450 1B1 (CYP) at concentrations (e.g., in vivo) effective for activating the deacetylase activity of the sirtuin. For instance, in preferred embodiments the sirtuin activator is chosen to have an EC₅₀ for activating sirtuin deacetylase activity that is at least 5 fold less than the EC₅₀ for inhibition of P450 1B1, and even more preferably at least 10 fold, 100 fold or even 1000 fold less.

In certain embodiments, the subject sirtuin activators do not have any substantial ability to inhibit nuclear factor-kappaB (NF-κB) at concentrations (e.g., in vivo) effective for activating the deacetylase activity of the sirtuin. For instance, in preferred embodiments the sirtuin activator is chosen to have an EC₅₀ for activating sirtuin deacetylase activity that is at least 5 fold less than the EC₅₀ for inhibition of NF-κB, and even more preferably at least 10 fold, 100 fold or even 1000 fold less.

In certain embodiments, the subject SIRT1 activators do not have any substantial ability to activate SIRT1 orthologs in lower eukaryotes, particularly yeast or human pathogens, at concentrations (e.g., in vivo) effective for activating the deacetylase activity of human SIRT1. For instance, in preferred embodiments the SIRT1 activator is chosen to have an EC50 for activating human SIRT1 deacetylase activity that is at least 5 fold less than the EC50 for activating yeast Sir2 (such as Candida, S. cerevisiae, etc), and even more preferably at least 10 fold, 100 fold or even 1000 fold less.

In other embodiments, the subject sirtuin activators do not have any substantial ability to inhibit protein kinases; to phosphorylate mitogen activated protein (MAP) kinases; to inhibit the catalytic or transcriptional activity of cyclo-oxygenases, such as COX-2; to inhibit nitric oxide synthase (iNOS); or to inhibit platelet adhesion to type I collagen at concentrations (e.g., in vivo) effective for activating the deacetylase activity of the sirtuin. For instance, in preferred embodiments, the sirtuin activator is chosen to have an EC₅₀ for activating sirtuin deacetylase activity that is at least 5 fold less than the EC₅₀ for performing any of these activities, and even more preferably at least 10 fold, 100 fold or even 1000 fold less.

In other embodiments, a compound described herein, e.g., a sirtuin activator or inhibitor, does not have significant or detectable anti-oxidant activities, as determined by any of the standard assays known in the art. For example, a compound does not significantly scavenge free-radicals, such as O₂ radicals. A compound may have less than about 2, 3, 5, 10, 30 or 100 fold anti-oxidant activity relative to another compound, e.g., resveratrol.

A compound may also have a binding affinity for a sirtuin of about 10⁻⁹M, 10⁻¹⁰M, 10⁻¹¹M, 10⁻¹²M or less. A compound may reduce the Kₘ of a sirtuin for its substrate or NAD⁺ by a factor of at least about 2, 3, 4, 5, 10, 20, 30, 50 or 100. A compound may have an EC₅₀ for activating the deacetylase activity of a sirtuin of less than about 1 nM, less than about 10 nM, less than about 100 nM, less than about 1 μM, less than about 10 μM, less than about 100 μM, or from about 1-10 nM, from about 10-100 nM, from about 0.1-1 μM, from about 1-10 μM or from about 10-100 μM. A compound may activate the deacetylase activity of a sirtuin by a factor of at least about 5, 10, 20, 30, 50, or 100, as measured in an acellular assay or in a cell based assay as described in the Examples. A compound may cause at least a 10%, 30%, 50%, 80%, 2 fold, 5 fold, 10 fold, 50 fold or 100 fold greater induction of the deacetylase activity of SIRT1 relative to the same concentration of resveratrol or other compound described herein. A compound may also have an EC₅₀ for activating SIRT5 that is at least about 10 fold, 20 fold, 30 fold, 50 fold greater than that for activating SIRT1.

A compound may traverse the cytoplasmic membrane of a cell. For example, a compound may have a cell-permeability of at least about 20%, 50%, 75%, 80%, 90% or 95%.

Compounds described herein may also have one or more of the following characteristics: the compound may be essentially non-toxic to a cell or subject; the compound may be an organic molecule or a small molecule of 2000 amu or less, 1000 amu or less; a compound may have a half-life under normal atmospheric conditions of at least about 30 days, 60 days, 120 days, 6 months or 1 year; the compound may have a half-life in solution of at least about 30 days, 60 days, 120 days, 6 months or 1 year; a compound may be more stable in solution than resveratrol by at least a factor of about 50%, 2 fold, 5 fold, 10 fold, 30 fold, 50 fold or 100 fold; a compound may promote deacetylation of the DNA repair factor Ku70; a compound may promote deacetylation of Re1A/p65; a compound may increase general turnover rates and enhance the sensitivity of cells to TNF-induced apoptosis.

An agent for use in the methods described herein may also be a protein, e.g., a sirtuin or a biologically active homolog thereof, or a nucleic acid encoding such. A nucleic acid may be linked to at least one regulatory element and may be part of a vector, e.g., an expression vector. The vector may target expression to a specific tissue, e.g., the colon, and may use a tissue specific promoter.

Other sirtuin inhibitors include siRNA molecules that specifically reduce the level of expression of sirtuins.

Compositions comprising at least 2, 3, 4, 5 or more compounds described herein are also provided, as well as compositions comprising 1, 2, 3, 4, 5 or more compounds described herein and other agents, e.g., chemotherapeutic agents, are also provided herein. The chemotherapeutic agents set forth in U.S. application having publication number 2006/0025337 are specifically incorporated by reference herein.

Methods of treatment or prevention described herein may be accompanied by a determination of the level and/or activity of β-catenin in a cell of the subject. This measurement may be conducted before, during, and/or after treatment by either methods described herein or alternative methods of treatment or prevention. In one embodiment, a biological sample is obtained from a subject and the level of activation of β-catenin is determined in the sample. Determining the level of activation of β-catenin may comprise determining the level of acetylation. If the measurement of β-catenin activity is performed in a subject that is not being treated, a higher level of acetylation relative to that in a control having a normal β-catenin activity level, indicates that the subject can be treated as described herein. If the measurement of β-catenin activity is performed in a subject that is already being treated, e.g., as described herein, a higher level of acetylation relative to that in a control having a normal β-catenin activity level, indicates that the treatment of the subject should be continued. If the measurement of β-catenin activity is performed in a subject in which the treatment is considered to have been concluded, a higher level of acetylation relative to that in a control having a normal β-catenin activity level, indicates that the treatment of the subject should be reinitiated.

A control against which a level of β-catenin activity is measured may comprise a statistical measure of the levels of β-catenin activity in a significant or sufficient number of subject that are not believed or known to have a disease described herein, e.g., subjects who are believed to be healthy. A statistically different, e.g., higher, level of β-catenin activity in a sample from a subject relative to a control would indicate that the subject can be treated as described herein.

In one embodiment, a sample of tissue, e.g., a tumor, of a subject is obtained, the level of activation of β-catenin therein is determined, and if the level of activation of β-catenin is elevated relative to a control level, the subject will be treated by administration of an agent that increases the level or activity of a sirtuin, e.g., SIRT1.

In one embodiment, a method is used for determining the likelihood of response of a subject having a disease associated with a dysregulated β-catenin activity, such as cancer, to a treatment with an agent that increases the level or activity of a sirtuin. A method may comprise determining the level of activity of β-catenin in a cell, e.g., a cancerous cell of the subject, wherein a higher level of β-catenin activity in the cancerous cell of the subject relative to that in a control indicates that the subject is likely to respond to the treatment.

Other methods provided herein are prognostic or predictive methods. A method may be for determining the prognosis of a subject having a disease, e.g., cancer, and being treated with an agent that increases the level or activity of a sirtuin. A method may comprise determining the level of activity of β-catenin in a cell, e.g., a cancerous cell, of the subject, wherein a level of β-catenin activity in the cancerous cell of the subject that is lower relative to that in the cancerous cell of the subject prior to the beginning of the treatment indicates that the subject is responsive to the treatment.

Also provided are methods for determining the prognosis of a subject having a disease, e.g., cancer, and being treated with an agent that increases the level or activity of a sirtuin. A method may comprise determining the cellular location of β-catenin in a cancerous cell of the subject, wherein the presence of β-catenin in a cell compartment other than the nucleus indicates that the subject is responsive to the treatment.

Furthermore, based at least on the observation that SIRT1 levels go up during calorie restriction (CR) and that SIRT1 overexpression at CR levels in a transgenic mouse slows β-catenin-driven tumor, a measurement of SIRT1 levels or activity in certain tissues of a subject may be predictive of the likelihood of the subject to develop a disease, e.g., cancer. In one embodiment, a method comprises determining the activity or level of a sirtuin, e.g., SIRT1, in a tissue of a subject, wherein a higher level or activity of the sirtuin indicates that the subject is less likely to develop cancer in the tissue than if the level or activity of the sirtuin was lower. A level of SIRT1 activity or protein level a tissue that is similar to a level that is observed under CR conditions in the tissue indicates that the likelihood of developing cancer in that tissue is lower than if the level of SIRT1 activity or protein level was lower. A level of SIRT1 in CR is about 50%, 2 fold, 3 fold, 5 fold or more higher than under non-CR conditions. In an illustrative embodiment, the level of SIRT1 activity or protein level is determined in a tissue sample from the intestines or the colon of a subject. A higher level of SIRT1 activity or protein relative to a control indicates that the subject is less likely to develop colon cancer than if the level was lower.

Prevention and Treatment of Human Diseases with SIRT1 Modulators

Misregulation of β-catenin activity results in disruption of the Wnt signaling pathway, which is reversed by sirtuins (e.g., SIRT1) and agents that modulate (e.g., increase) the level or activity of a sirtuin. Therefore, sirtuin modulators are useful in treating Wnt-signaling associated diseases. Exemplary Wnt-signaling associated diseases are provided below in Table D1 and the references therein, which are incorporated herein by reference in their entireties. See also Moon et al., Nat Rev Genet. (2004) 5(9):691-701.

TABLE D1

| Gene | Disease | References: |
|---|---|---|
| APC | Polyposis coli | Kinzler KW, et al.; Science. (1991); 253(5020): 661-5 |
| | | Nishisho I, et al.; Science. (1991); 253(5020): 665-9 |
| LRP5 | Bone Density defects | Gong, Y, et al.; Cell.(2001); 107(4): 513-23 |
| | Vascular defects in the eye | Little, RD, et al.; Am J Hum Genet. (2002); 70(1): 11-9. |
| | (Osteoperosis-pseudoglioma | Boyden, LM, et al.; N Engl J Med. (2002); |
| | Syndrome, OPPG) | 346(20): 1513-21 |
| LRP5 | Familial Exudative | Toomes, C., et al.; Am J Hum Genet. (2004); 74(4): 721-30. |
| | Vitreoretinopathy | Qin, M., et al.; Hum Mutat. (2005); 26(2): 104-12 |
| LRP6 | early coronary disease | Mani, A, et al., Science (2007); 315: 1278-92. |
| LRP6 | Late onset Alzheimer | De Ferrari GV, et al.; Proc Natl Acad Sci. USA. |
| | | (2007); 104(22): 9434-9. |
| FZD4 | Familial Exudative | Robitaille, J. et al.; Nat Genet. (2002); 32(2): 326-30. |
| | Vitreoretinopathy: | Qin, M. et al.; Hum Mutat. (2005); (2): 104-12 |
| | retinal angiogenesis | |
| Norrin | Familial Exudative | Xu, Q, et al.; Cell. (2004); 116(6): 883-95. |
| | Vitreoretinopathy | |
| WNT3 | Tetra-Amelia | Neimann, S., et al.; Am J Hum Genet. (2004); |
| | | 74(3): 558-63. |
| WNT4 | Mullerian-duct regression and | Biason-Lauber, A.; et al.; N Engl J Med. (2004); |
| | virilization | 351(8): 792-8. |
| WNT5B | Type II diabetes | Kanazawa, A, et al.; Am J Hum Genet. (2004); |
| | | 75(5): 832-43. |
| WNT7A | Fuhrmann syndrome | Woods, CJ, et al.; Am J Hum Genet. (2006); 79(2): 402-8. |
| WNT10A | Odonto-onycho-dermal | Adaimy, L., et al.; Am J Hum Genet. (2007); 81(4): 821-8. |
| | dysplasia | |
| WNT10B | Obesity | Christodoulides, C., et al.; Diabetologia. (2006); |
| | | 49(4): 678-84. |
| AXIN1 | caudal duplication | Oates, NA, et al.; Am J Hum Genet. (2006); 79(1): 155-62. |
| TCF7L2 | Type II diabetes | Grant, S. F., et al.; Nat Genet. (2006); 38(3): 320-3. |
| (TCF4) | | Florez, JC, et al.; N Engl J Med. (2006); 355(3): 241-50. |
| | | O'Rahilly, S. and Wareham, NJ; N Engl J Med. (2006); |
| | | 355(3): 306-8. |
| AXIN2 | Tooth agenesis | Lammli, et al.; Am J Hum Genet. (2004); 74(5): 1043-50. |
| WTX | Wilms tumor | Major, MD, et al.; Science. (2007); 316(5827): 1043-6. |
| | | Rivera, MN, et al.; Science. (2007); 315(5812): 642-5. |
| PORC1 | Focal dermal hypoplasia | Grzeschik, KH, et al.; Nat Genet. (2007); 39(7): 833-5. |
| | | Wang, X., et al., Nat Genet. (2007); 39(7): 836-8. |
| RSPO4 | autosomal recessive | Bergmann, C., et al.; Am J Hum Genet. (2006); |
| | anonychia | 79(6): 1105-9 Blaydon, DC., et al.; Nat Genet. (2006); |
| | | 38(11): 1245-7. |
| VANGL1 | Neural tube defects | Kibar, Z., et al.; N Engl J Med. (2007); 356(14): 1432-7. |

In some embodiments, a method of treating a Wnt signaling-associated disease includes administering to a mammalian subject, such as a human, in need thereof a therapeutically effective amount of an agent that increases the level or activity of a sirtuin, e.g., SIRT1. The mammalian subject may be clinically diagnosed as having the disease. Also, the subject may have an abnormal level of beta-catenin activation in one or more cells, tissues or organs of interest. Treatment may result in reversal of the disease, or the alleviation of one or more symptoms of the disease. Alternatively, progression of the disease may be stopped, or the rate of progression reduced. Efficacy may be determined using methods known to those skilled in the art, and may be determined relative to non-treatment of the disease, or the treatment of the disease with other compounds. Treatment of a subject may be performed in combination with another treatment modality. For example, treatment of a subject suffering from cancer may include treatment of a sirtuin modulator and a cell cycle-specific cytoreductive therapy, e.g. chemotherapy with S-phase specific agents, and radiation therapy.

In other embodiments, the invention provides a method of preventing a human subject from developing a Wnt signaling-associated disease includes administering to a mammalian subject, such as a human, in need thereof an effective amount of an agent that increases the level or activity of a sirtuin, e.g., SIRT1. The mammalian subject may be clinically diagnosed as having a risk of developing the disease, or may be at increased risk of developing the disease based on the presence of one or more gene mutations, such as in a gene listed above in Table D1, in the subject or in the subject's family Also, the subject may have an abnormal level of beta-catenin activation in one or more cells, tissues or organs of interest.

### Other Methods

Additional methods that are provided herein include methods for modulating β-catenin activity. A method may comprise modulating β-catenin acetylation levels. Methods may be, e.g., in vitro, in vivo, in situ or ex vivo. In one embodiment, a method comprises contacting a β-catenin protein or homolog thereof with a sirtuin or biologically active homolog thereof under conditions in which the sirtuin or biologically active homolog thereof deacetylates the β-catenin protein or homolog thereof, to thereby reduce β-catenin acetylation levels and β-catenin activity. A method may also comprise contacting a cell comprising a β-catenin protein or homolog thereof, which is either endogenous or heterologous, with a sirtuin or biologically active homolog thereof, or nucleic acid encoding such, or agent activating a sirtuin. Exemplary agents are those further described herein.

Methods for preventing the deacetylation of β-catenin, to thereby prevent loss of β-catenin activity are also provided. A method may comprise contacting a solution, extract, cell extract or cell comprising a β-catenin protein with an agent that inhibits the activity or decreases the levels of a sirtuin, e.g., SIRT1. Exemplary agents are further described herein.

Modulation of the activity of β-catenin proteins may also modulate biological activities that are mediated or associated with β-catenin activity. Thus, the methods described herein for modulating β-catenin activity may be used for modulating β-catenin-driven proliferation of a cell. A method may comprise providing a cell whose proliferation is driven by β-catenin; and contacting the cell with an agent that increases sirtuin level or activity. A method may also comprise providing a cell; determining whether the proliferation of the cell is driven by β-catenin; and contacting a cell whose proliferation is driven by β-catenin with an agent that increases sirtuin level or activity.

### Screening Assays

Based at least on the observation that SIRT1 deacetylates β-catenin and thereby inactivates β-catenin, methods for identifying agents, e.g., small molecules, that modulate β-catenin activity can be formulated.

Certain methods may comprise identifying an agent that modulates the interaction between a sirtuin and a β-catenin protein. An exemplary method comprises contacting a sirtuin or homolog thereof sufficient for interacting with a β-catenin protein with a β-catenin protein or homolog thereof sufficient to interact with a sirtuin and with a test agent under conditions in which the sirtuin or homolog thereof and the β-catenin or homolog thereof in the presence relative to the absence of the test agent interact in the absence of the test agent, wherein a difference in the level of interaction between the sirtuin or homolog thereof and the β-catenin or homolog thereof indicates that the test agent modulates the interaction. The method may be a method for identifying an agent that inhibits the interaction between a sirtuin and a β-catenin protein, wherein a lower level of interaction between the sirtuin or homolog thereof and the β-catenin or homolog thereof indicates that the test agent inhibits the interaction. The method may be a method for identifying an agent that stimulates the interaction between a sirtuin and a β-catenin protein, wherein a higher level of interaction between the sirtuin or homolog thereof and the β-catenin or homolog thereof indicates that the test agent stimulates the interaction.

Certain methods can be used for identifying an agent that modulates the deacetylation of β-catenin by a sirtuin. A method may comprise contacting a sirtuin or homolog thereof sufficient for deacetylating a β-catenin protein with a β-catenin protein or homolog thereof sufficient to be deacetylated by a sirtuin and with a test agent under conditions in which the sirtuin or homolog thereof deacetylates the β-catenin or homolog thereof in the absence of the test agent, wherein a difference in the level of acetylation of the β-catenin protein or homolog thereof in the presence relative to the absence of the test agent indicates that the test agent modulates the deacetylation of β-catenin by the sirtuin. The method may be a method for identifying an agent that inhibits the deacetylation of a β-catenin protein, wherein a higher level of acetylation of the β-catenin protein or homolog thereof in the presence relative to the absence of the test agent indicates that the test agent inhibits the deacetylation (or promotes or maintains acetylation) of β-catenin by the sirtuin. The method may be a method for identifying an agent that stimulates the deacetylation of a β-catenin protein, wherein a lower level of acetylation of the β-catenin protein or homolog thereof in the presence relative to the absence of the test agent indicates that the test agent stimulates the deacetylation (or inhibits acetylation) of β-catenin by the sirtuin.

Interaction between two proteins may be detected by a variety of techniques. Modulation of the formation of complexes can be quantitated using, for example, detectably labeled proteins such as radiolabelled, fluorescently labeled, or enzymatically labeled polypeptides, by immunoassay, by chromatographic detection, or by detecting the intrinsic activity of the acetyl transferase or deacetylase.

Typically, it will be desirable to immobilize one of the proteins to facilitate separation of complexes from uncomplexed forms of one or both of the proteins, as well as to accommodate automation of the assay. Binding of the proteins, in the presence and absence of a candidate agent, can be accomplished in any vessel suitable for containing the reactants. Examples include microtitre plates, test tubes, and micro-centrifuge tubes.

In one embodiment, a sirtuin or homolog thereof and/or a β-catenin protein or homolog thereof is provided in the form of a fusion protein comprising a domain that allows the protein to be bound to a matrix. For example, glutathione-S-transferase fusion proteins can be adsorbed onto glutathione sepharose beads (Sigma Chemical, St. Louis, Mo.) or glutathione derivatized microtitre plates, which are then combined with the other protein, which may be labeled, and the test compound, and the mixture incubated under conditions conducive to complex formation, e.g. at physiological conditions for salt and pH, though slightly more stringent conditions may be desired. Following incubation, the beads may be washed to remove any unbound label, the matrix immobilized and the presence of radiolabel determined directly (e.g. beads placed in scintillant), or in the supernatant after the complexes are subsequently dissociated. Alternatively, the complexes can be dissociated from the matrix, separated by SDS-PAGE, and the level of binding protein found in the bead fraction quantitated from the gel using standard electrophoretic techniques.

Other techniques for immobilizing proteins or peptides on matrices are also available for use in the subject assay. For instance, a protein can be immobilized utilizing conjugation of biotin and streptavidin. For instance, biotinylated sirtuin or β-catenin molecules can be prepared from biotin-NHS (N-hydroxy-succinimide) using techniques well known in the art (e.g., biotinylation kit, Pierce Chemicals, Rockford, Ill.), and immobilized in the wells of streptavidin-coated 96 well plates (Pierce Chemical). Alternatively, antibodies reactive with either acetylated or deacetylated β-catenin proteins or portions thereof, but which preferably do not interfere with the interaction between the β-catenin molecule and the binding protein, can be derivatized to the wells of the plate, and β-catenin trapped in the wells by antibody conjugation. As above, preparations of an binding protein and a test compound are incubated in the β-catenin-presenting wells of the plate, and the amount of complex trapped in the well can be quantitated. Exemplary methods for detecting such complexes, in addition to those described above for the GST-immobilized complexes, include immunodetection of complexes using antibodies reactive with the binding protein, or which are reactive with β-catenin protein and compete with the binding protein; as well as enzyme-linked assays which rely on detecting an enzymatic activity associated with the binding protein, either intrinsic or extrinsic activity. In the instance of the latter, the enzyme can be chemically conjugated or provided as a fusion protein with the binding protein. To illustrate, the binding protein can be chemically cross-linked or genetically fused (if it is a polypeptide) with horseradish peroxidase, and the amount of polypeptide trapped in the complex can be assessed with a chromogenic substrate of the enzyme, e.g. 3,3′-diamino-benzadine terahydrochloride or 4-chloro-1-napthol. Likewise, a fusion protein comprising the polypeptide and glutathione-S-transferase can be provided, and complex formation quantitated by detecting the GST activity using 1-chloro-2,4-dinitrobenzene (Habig et al (1974) J Biol Chem 249:7130).

For processes which rely on immunodetection for quantitating proteins trapped in the complex, antibodies against the protein, such as anti-β-catenin, antibodies, can be used. Such antibodies can be obtained from various commercial vendors, e.g., as described elsewhere herein. Alternatively, the protein to be detected in the complex can be “epitope tagged” in the form of a fusion protein which includes, in addition to the β-catenin sequence, a second polypeptide for which antibodies are readily available (e.g. from commercial sources). For instance, the GST fusion proteins described above can also be used for quantification of binding using antibodies against the GST moiety. Other useful epitope tags include myc-epitopes (e.g., see Ellison et al. J Biol Chem. 266:21150-21157 (1991)) which includes a 10-residue sequence from c-myc, as well as the pFLAG system (International Biotechnologies, Inc.) or the pEZZ-protein A system (Pharmacia, N.J.).

The efficacy of a test compound can be assessed by generating dose response curves from data obtained using various concentrations of the test compound. Moreover, a control assay can also be performed to provide a baseline for comparison. In an exemplary control assay, interaction of a β-catenin protein or homolog thereof and a sirtuin protein or homolog thereof is quantitated in the absence of the test compound.

In a certain embodiment, a method for identifying an agent that modulates the activity of β-catenin may comprise contacting a cell or cell extract or cell lystate comprising one or more heterologous nucleic acids encoding a sirtuin or a homolog thereof that binds to β-catenin and/or β-catenin or a homolog thereof that binds to a sirtuin with a test agent; and determining the activity of β-catenin, wherein a different activity of β-catenin in a cell or cell extract or cell lysate that was contacted with the test agent relative to a cell, cell extract or cell lysate that was not contacted with a test agent indicates that the test agent is an agent that modulates the activity of β-catenin.

A method for identifying an agent that modulates the activity of β-catenin may also comprise contacting a cell comprising one or more heterologous nucleic acids encoding a sirtuin or a homolog thereof that binds to β-catenin and/or β-catenin or a homolog thereof that binds to a sirtuin with a test agent; and determining the cellular location of β-catenin, wherein a cellular location other than nuclear in a cell that was contacted with the test agent indicates that the test agent is an agent that modulates the activity of β-catenin.

Determining the activity of a β-catenin protein or homolog thereof may comprise determining a biological activity that is mediated by β-catenin activity, such as cell proliferation.

Various methods or steps thereof, such as those described herein, may also be combined. Any of the screening assays described herein may further comprise determining the effect of a test compound on tumor size or growth, such as by using animal models, e.g., nude mice.

### Pharmaceutical Compositions and Methods

Pharmaceutical compositions for use in accordance with the present methods may be formulated in conventional manner using one or more physiologically acceptable carriers or excipients. Thus, compounds, e.g., sirtuin activating compounds, and their physiologically acceptable salts and solvates may be formulated for administration by, for example, injection, inhalation or insufflation (either through the mouth or the nose) or oral, buccal, parenteral or rectal administration. In one embodiment, the compound is administered locally, at the site where the target cells, e.g., diseased cells, are present, i.e., in the blood or in a joint.

Compounds can be formulated for a variety of loads of administration, including systemic and topical or localized administration. Techniques and formulations generally may be found in Remmington's Pharmaceutical Sciences, Meade Publishing Co., Easton, Pa. For systemic administration, injection is preferred, including intramuscular, intravenous, intraperitoneal, and subcutaneous. For injection, the compounds can be formulated in liquid solutions, preferably in physiologically compatible buffers such as Hank's solution or Ringer's solution. In addition, the compounds may be formulated in solid form and redissolved or suspended immediately prior to use. Lyophilized forms are also included.

For oral administration, the pharmaceutical compositions may take the form of, for example, tablets, lozanges, or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., ationd oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate. Preparations for oral administration may be suitably formulated to give controlled release of the active compound.

For administration by inhalation, the compounds may be conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin, for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

Pharmaceutical compositions (including cosmetic preparations) may comprise from about 0.00001 to 100% such as from 0.001 to 10% or from 0.1% to 5% by weight of one or more compounds described herein.

In one embodiment, a compound described herein, is incorporated into a topical formulation containing a topical carrier that is generally suited to topical drug administration and comprising any such material known in the art. The topical carrier may be selected so as to provide the composition in the desired form, e.g., as an ointment, lotion, cream, microemulsion, gel, oil, solution, or the like, and may be comprised of a material of either naturally occurring or synthetic origin. It is preferable that the selected carrier not adversely affect the active agent or other components of the topical formulation. Examples of suitable topical carriers for use herein include water, alcohols and other nontoxic organic solvents, glycerin, mineral oil, silicone, petroleum jelly, lanolin, fatty acids, vegetable oils, parabens, waxes, and the like.

Formulations may be colorless, odorless ointments, lotions, creams, microemulsions and gels.

Compounds may be incorporated into ointments, which generally are semisolid preparations which are typically based on petrolatum or other petroleum derivatives. The specific ointment base to be used, as will be appreciated by those skilled in the art, is one that will provide for optimum drug delivery, and, preferably, will provide for other desired characteristics as well, e.g., emolliency or the like. As with other carriers or vehicles, an ointment base should be inert, stable, nonirritating and nonsensitizing. As explained in Remington's, cited in the preceding section, ointment bases may be grouped in four classes: oleaginous bases; emulsifiable bases; emulsion bases; and water-soluble bases. Oleaginous ointment bases include, for example, vegetable oils, fats obtained from animals, and semisolid hydrocarbons obtained from petroleum. Emulsifiable ointment bases, also known as absorbent ointment bases, contain little or no water and include, for example, hydroxystearin sulfate, anhydrous lanolin and hydrophilic petrolatum. Emulsion ointment bases are either water-in-oil (W/O) emulsions or oil-in-water (O/W) emulsions, and include, for example, cetyl alcohol, glyceryl monostearate, lanolin and stearic acid. Exemplary water-soluble ointment bases are prepared from polyethylene glycols (PEGs) of varying molecular weight; again, reference may be had to Remington's, supra, for further information.

Compounds may be incorporated into lotions, which generally are preparations to be applied to the skin surface without friction, and are typically liquid or semiliquid preparations in which solid particles, including the active agent, are present in a water or alcohol base. Lotions are usually suspensions of solids, and may comprise a liquid oily emulsion of the oil-in-water type. Lotions are preferred formulations for treating large body areas, because of the ease of applying a more fluid composition. It is generally necessary that the insoluble matter in a lotion be finely divided. Lotions will typically contain suspending agents to produce better dispersions as well as compounds useful for localizing and holding the active agent in contact with the skin, e.g., methylcellulose, sodium carboxymethylcellulose, or the like. An exemplary lotion formulation for use in conjunction with the present method contains propylene glycol mixed with a hydrophilic petrolatum such as that which may be obtained under the trademark Aquaphor® from Beiersdorf, Inc. (Norwalk, Conn.).

Compounds may be incorporated into creams, which generally are viscous liquid or semisolid emulsions, either oil-in-water or water-in-oil. Cream bases are water-washable, and contain an oil phase, an emulsifier and an aqueous phase. The oil phase is generally comprised of petrolatum and a fatty alcohol such as cetyl or stearyl alcohol; the aqueous phase usually, although not necessarily, exceeds the oil phase in volume, and generally contains a humectant. The emulsifier in a cream formulation, as explained in Remington's, supra, is generally a nonionic, anionic, cationic or amphoteric surfactant.

Compounds may be incorporated into microemulsions, which generally are thermodynamically stable, isotropically clear dispersions of two immiscible liquids, such as oil and water, stabilized by an interfacial film of surfactant molecules (Encyclopedia of Pharmaceutical Technology (New York: Marcel Dekker, 1992), volume 9). For the preparation of microemulsions, surfactant (emulsifier), co-surfactant (co-emulsifier), an oil phase and a water phase are necessary. Suitable surfactants include any surfactants that are useful in the preparation of emulsions, e.g., emulsifiers that are typically used in the preparation of creams. The co-surfactant (or “co-emulsifer”) is generally selected from the group of polyglycerol derivatives, glycerol derivatives and fatty alcohols. Preferred emulsifier/co-emulsifier combinations are generally although not necessarily selected from the group consisting of: glyceryl monostearate and polyoxyethylene stearate; polyethylene glycol and ethylene glycol palmitostearate; and caprilic and capric triglycerides and oleoyl macrogolglycerides. The water phase includes not only water but also, typically, buffers, glucose, propylene glycol, polyethylene glycols, preferably lower molecular weight polyethylene glycols (e.g., PEG 300 and PEG 400), and/or glycerol, and the like, while the oil phase will generally comprise, for example, fatty acid esters, modified vegetable oils, silicone oils, mixtures of mono- di- and triglycerides, mono- and di-esters of PEG (e.g., oleoyl macrogol glycerides), etc.

Compounds may be incorporated into gel formulations, which generally are semisolid systems consisting of either suspensions made up of small inorganic particles (two-phase systems) or large organic molecules distributed substantially uniformly throughout a carrier liquid (single phase gels). Single phase gels can be made, for example, by combining the active agent, a carrier liquid and a suitable gelling agent such as tragacanth (at 2 to 5%), sodium alginate (at 2-10%), gelatin (at 2-15%), methylcellulose (at 3-5%), sodium carboxymethylcellulose (at 2-5%), carbomer (at 0.3-5%) or polyvinyl alcohol (at 10-20%) together and mixing until a characteristic semisolid product is produced. Other suitable gelling agents include methylhydroxycellulose, polyoxyethylene-polyoxypropylene, hydroxyethylcellulose and gelatin. Although gels commonly employ aqueous carrier liquid, alcohols and oils can be used as the carrier liquid as well.

Various additives, known to those skilled in the art, may be included in formulations, e.g., topical formulations. Examples of additives include, but are not limited to, solubilizers, skin permeation enhancers, opacifiers, preservatives (e.g., anti-oxidants), gelling agents, buffering agents, surfactants (particularly nonionic and amphoteric surfactants), emulsifiers, emollients, thickening agents, stabilizers, humectants, colorants, fragrance, and the like. Inclusion of solubilizers and/or skin permeation enhancers is particularly preferred, along with emulsifiers, emollients and preservatives. An optimum topical formulation comprises approximately: 2 wt. % to 60 wt. %, preferably 2 wt. % to 50 wt. %, solubilizer and/or skin permeation enhancer; 2 wt. % to 50 wt. %, preferably 2 wt. % to 20 wt. %, emulsifiers; 2 wt. % to 20 wt. % emollient; and 0.01 to 0.2 wt. % preservative, with the active agent and carrier (e.g., water) making of the remainder of the formulation.

A skin permeation enhancer serves to facilitate passage of therapeutic levels of active agent to pass through a reasonably sized area of unbroken skin. Suitable enhancers are well known in the art and include, for example: lower alkanols such as methanol ethanol and 2-propanol; alkyl methyl sulfoxides such as dimethylsulfoxide (DMSO), decylmethylsulfoxide (C.sub.10 MSO) and tetradecylmethyl sulfboxide; pyrrolidones such as 2-pyrrolidone, N-methyl-2-pyrrolidone and N-(-hydroxyethyl)pyrrolidone; urea; N,N-diethyl-m-toluamide; C.sub.2 -C.sub.6 alkanediols; miscellaneous solvents such as dimethyl formamide (DMF), N,N-dimethylacetamide (DMA) and tetrahydrofurfuryl alcohol; and the 1-substituted azacycloheptan-2-ones, particularly 1-n-dodecylcyclazacycloheptan-2-one (laurocapram; available under the trademark Azone® from Whitby Research Incorporated, Richmond, Va.).

Examples of solubilizers include, but are not limited to, the following: hydrophilic ethers such as diethylene glycol monoethyl ether (ethoxydiglycol, available commercially as Transcutol®) and diethylene glycol monoethyl ether oleate (available commercially as Softcutol®); polyethylene castor oil derivatives such as polyoxy 35 castor oil, polyoxy 40 hydrogenated castor oil, etc.; polyethylene glycol, particularly lower molecular weight polyethylene glycols such as PEG 300 and PEG 400, and polyethylene glycol derivatives such as PEG-8 caprylic/capric glycerides (available commercially as Labrasol®); alkyl methyl sulfoxides such as DMSO; pyrrolidones such as 2-pyrrolidone and N-methyl-2-pyrrolidone; and DMA. Many solubilizers can also act as absorption enhancers. A single solubilizer may be incorporated into the formulation, or a mixture of solubilizers may be incorporated therein.

Suitable emulsifiers and co-emulsifiers include, without limitation, those emulsifiers and co-emulsifiers described with respect to microemulsion formulations. Emollients include, for example, propylene glycol, glycerol, isopropyl myristate, polypropylene glycol-2 (PPG-2) myristyl ether propionate, and the like.

Other active agents may also be included in formulations, e.g., other anti-inflammatory agents, analgesics, antimicrobial agents, antifungal agents, antibiotics, vitamins, antioxidants, and sunblock agents commonly found in sunscreen formulations including, but not limited to, anthranilates, benzophenones (particularly benzophenone-3), camphor derivatives, cinnamates (e.g., octyl methoxycinnamate), dibenzoyl methanes (e.g., butyl methoxydibenzoyl methane), p-aminobenzoic acid (PABA) and derivatives thereof, and salicylates (e.g., octyl salicylate).

In certain topical formulations, the active agent is present in an amount in the range of approximately 0.25 wt. % to 75 wt. % of the formulation, preferably in the range of approximately 0.25 wt. % to 30 wt. % of the formulation, more preferably in the range of approximately 0.5 wt. % to 15 wt. % of the formulation, and most preferably in the range of approximately 1.0 wt. % to 10 wt. % of the formulation.

Topical skin treatment compositions can be packaged in a suitable container to suit its viscosity and intended use by the consumer. For example, a lotion or cream can be packaged in a bottle or a roll-ball applicator, or a propellant-driven aerosol device or a container fitted with a pump suitable for finger operation. When the composition is a cream, it can simply be stored in a non-deformable bottle or squeeze container, such as a tube or a lidded jar. The composition may also be included in capsules such as those described in U.S. Pat. No. 5,063,507. Accordingly, also provided are closed containers containing a cosmetically acceptable composition as herein defined.

In an alternative embodiment, a pharmaceutical formulation is provided for oral or parenteral administration, in which case the formulation may comprises an activating compound-containing microemulsion as described above, but may contain alternative pharmaceutically acceptable carriers, vehicles, additives, etc. particularly suited to oral or parenteral drug administration. Alternatively, an activating compound-containing microemulsion may be administered orally or parenterally substantially as described above, without modification.

Phospholipids complexes, e.g., resveratrol-phospholipid complexes, and their preparation are described in US2004116386. Methods for stabilizing active components using polyol/polymer microcapsules, and their preparation are described in US20040108608. Processes for dissolving lipophilic compounds in aqueous solution with amphiphilic block copolymers are described in WO 04/035013.

Conditions of the eye can be treated or prevented by, e.g., systemic, topical, intraocular injection of a compound described herein, or by insertion of a sustained release device that releases a compound described herein.

Compounds described herein may be stored in oxygen free environment according to methods in the art. For example, resveratrol or analog thereof can be prepared in an airtight capusule for oral administration, such as Capsugel from Pfizer, Inc.

Cells, e.g., treated ex vivo with a compound described herein, can be administered according to methods for administering a graft to a subject, which may be accompanied, e.g., by administration of an immunosuppressant drug, e.g., cyclosporin A. For general principles in medicinal formulation, the reader is referred to Cell Therapy: Stem Cell Transplantation, Gene Therapy, and Cellular Immunotherapy, by G. Morstyn & W. Sheridan eds, Cambridge University Press, 1996; and Hematopoietic Stem Cell Therapy, E. D. Ball, J. Lister & P. Law, Churchill Livingstone, 2000.

Toxicity and therapeutic efficacy of compounds can be determined by standard pharmaceutical procedures in cell cultures or experimental animals. The LD₅₀ is the dose lethal to 50% of the population). The ED50 is the dose therapeutically effective in 50% of the population. The dose ratio between toxic and therapeutic effects (LD₅₀/ED₅₀) is the therapeutic index. Compounds that exhibit large therapeutic indexes are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds may lie within a range of circulating concentrations that include the ED₅₀ with little or no toxicity. The dosage may vary within this range depending upon the dosage form employed and the route of administration utilized. For any compound, the therapeutically effective dose can be estimated initially from cell culture assays. A dose may be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography.

Methods for increasing the protein level of a sirtuin in a cell may also comprise increasing the level of expression of the gene. In addition, one or more nucleic acids encoding a sirtuin may be introduced into a cell to increase the level of the sirtuin protein in the cell. In an exemplary embodiment, a vector encoding a sirtuin is introduced into a cell. A vector may be a viral vector. Viral vectors for administering to subjects are well known in the art, and include adenoviral vectors. For example, the transgene may be incorporated into any of a variety of viral vectors useful in gene therapy, such as recombinant retroviruses, adenovirus, adeno-associated virus (AAV), and herpes simplex virus-1, or recombinant bacterial or eukaryotic plasmids. While various viral vectors may be used in the practice of the methods described herein, AAV- and adenovirus-based approaches are of particular interest. Such vectors are generally understood to be the recombinant gene delivery system of choice for the transfer of exogenous genes in vivo, particularly into humans.

It is possible to limit the infection spectrum of viruses by modifying the viral packaging proteins on the surface of the viral particle (see, for example PCT publications WO93/25234, WO94/06920, and WO94/11524). For instance, strategies for the modification of the infection spectrum of viral vectors include: coupling antibodies specific for cell surface antigens to envelope protein (Roux et al., (1989) PNAS USA 86:9079-9083; Julan et al., (1992) J. Gen Virol 73:3251-3255; and Goud et al., (1983) Virology 163:251-254); or coupling cell surface ligands to the viral envelope proteins (Neda et al., (1991) J. Biol. Chem. 266:14143-14146). Coupling can be in the form of the chemical cross-linking with a protein or other variety (e.g. lactose to convert the env protein to an asialoglycoprotein), as well as by generating fusion proteins (e.g. single-chain antibody/env fusion proteins). This technique, while useful to limit or otherwise direct the infection to certain tissue types, and can also be used to convert an ecotropic vector in to an amphotropic vector.

Nucleic acids and proteins can also be administered in a form of a complex with other components, e.g., agents facilitating delivery to the target tissue or organ, agents facilitating transport through the cell membrane or the gut. For example proteins may be in the form of fusion proteins, fused, e.g., to transcytosis peptides. Nucleic acids and proteins may be administered with liposomes.

Administration of a sirtuin activator or other agent that increases the activity or protein level of a sirtuin may be followed by measuring a factor in the subject, such as measuring the activity of the sirtuin. In an illustrative embodiment, a cell is obtained from a subject following administration of an activating compound to the subject, such as by obtaining a biopsy, and the activity of the sirtuin or sirtuin expression level is determined in the biopsy. Alternatively, biomarkers, such as plasma biomarkers may be followed. The cell may be any cell of the subject, but in cases in which an activating compound is administered locally, the cell is preferably a cell that is located in the vicinity of the site of administration.

### Kits

Also provided herein are kits, e.g., kits for therapeutic purposes or kits for screening assays. A kit may comprise one or more activating or inhibitory compounds described herein, e.g., in premeasured doses. A kit may optionally comprise devices for contacting cells with the compounds and instructions for use. Devices include syringes, stents and other devices for introducing a compound into a subject or applying it to the skin of a subject.

Further, a kit may also contain components for measuring a factor, e.g., described above, such as the activity of sirtuin proteins, e.g., in tissue samples.

Other kits include kits for diagnosing the likelihood of having or developing a disorder. A kit may comprise an agent for measuring the activity and or expression level of a sirtuin.

Kits for screening assays are also provided. Exemplary kits comprise one or more agents for conducting a screening assay, such as a sirtuin, or a biologically active portion thereof, or a cell or cell extract comprising such. Any of the kits may also comprise instructions for use.

The invention now being generally described, it will be more readily understood by reference to the following examples, which are included merely for purposes of illustration of certain aspects and embodiments of the present invention, and are not intended to limit the invention.

### Examples

### Example 1

### SIRT1 Mimics the Ability of CR to Suppress Colon Cancer

Caloric restriction (CR) is one of the most effective ways to extend lifespan and reduce spontaneous tumors in mammals, yet the mechanism is unknown. SIRT1 is an NAD⁺-dependent deacetylase proposed to underlie the health benefits of CR. Here we show that SIRT1 is more highly expressed in the intestines of rodents on a CR diet and that overexpression of SIRT1 in the gut of a tumor prone mouse model suppresses intestinal tumor growth and morbidity, mirroring the beneficial effect of CR in this model. We find that SIRT1 interacts with and deacetylates the oncogenic form of β-catenin resulting in suppression of β-catenin driven transcription and reduced cellular proliferation. These findings implicate SIRT1 and the β-catenin pathway as important effectors of the cancer preventive effects of CR and suggest new approaches to treating a variety of human cancers.

SIRT1 deacetylates and represses β-catenin activity thereby suppressing tumorigenesis in the APC^{min/+} mouse model when expressed in the gut at levels that mimic CR.

In lower species, the SIR2 gene is proposed to mediate lifespan extension by CR (1). The human Sir2 gene family is comprised of seven members, SIRT1-7. SIRT1, the best-characterized sirtuin, is induced by CR and regulates such processes as insulin production and fat metabolism, leading to speculation that sirtuins might also mediate the effects of CR in mammals (for review see (2)). CR is known to inhibit cancer but existing data is conflicting as to whether SIRT1 mediates this protective effect (3). For example, SIRT1 is expressed highly in tumors lacking HIC1 (4), inhibits apoptosis (5-7), and down-regulates the expression of tumor suppressor genes, leading many to conclude that SIRT1 is an oncogene (4, 8). On the other hand, SIRT1 can be pro-apoptotic (9) and anti-proliferative (10, 11), consistent with it having tumor suppressor activity. This question is important to resolve, particularly given the overarching question as to whether longevity-promoting genes in lower organisms function as oncogenes or tumor suppressors in mammals.

To help resolve this debate, we tested whether upregulating SIRT1 in a mouse cancer model can recapitulate the tumor suppressive effect of CR. We chose to analyze the APC^{min/+} model of colon cancer for a variety of reasons: it recapitulates many aspects of the colon cancer in humans, the mechanism of tumorigenesis is well characterized, and CR reduces the rate of tumorigenesis by about 4-fold (12).

The APC^{min/+} mouse contains a germline mutation in the tumor suppressor APC (adenomatous polyposis coli). Somatic loss of the second allele leads to constitutive nuclear localization of β-catenin and adenomatous polyp formation (13). β-catenin is a key effector of the Wnt signaling pathway and plays a significant role in stem cell maintenance, development and carcinogenesis (14). Constitutive activation of the β-catenin pathway has been found in 90% of colorectal cancers. In addition, it is aberrantly activated in many other aging related cancers such as prostate, breast and melanoma.

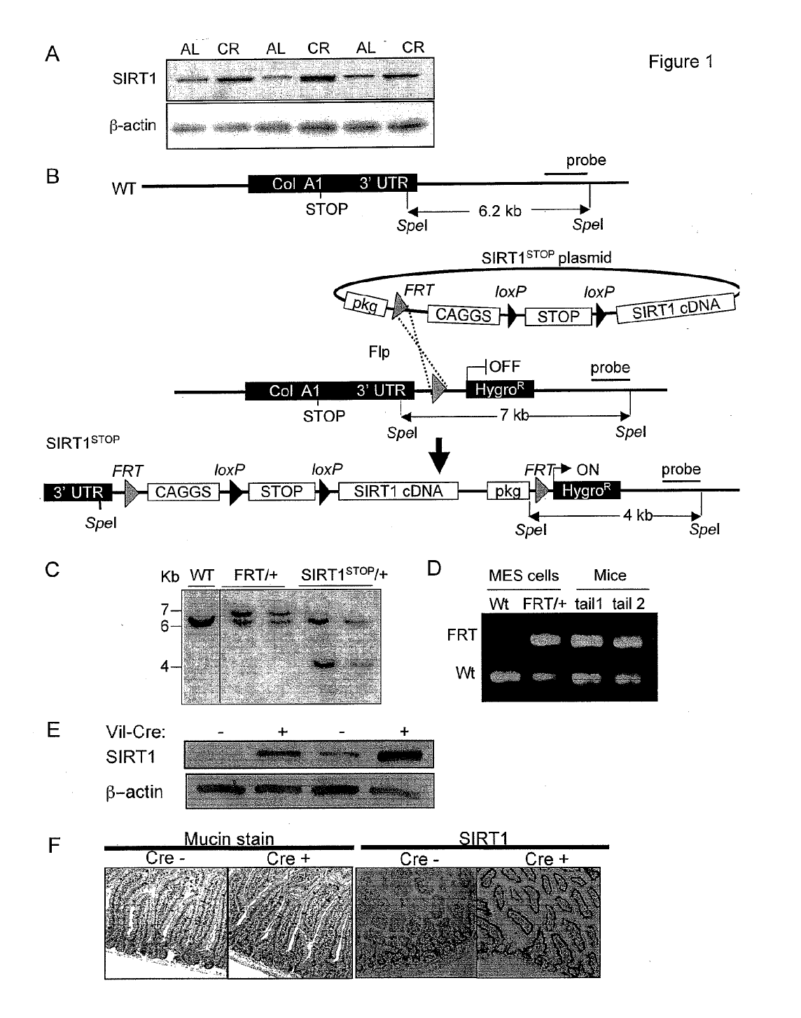

We observed that rodents on a CR diet had a 2-fold higher level of SIRT1 protein in the gut epithelium relative to ad lib-fed controls (FIG. 1A). To mimic this level of SIRT1 upregulation in gut epithelial cells, we generated a floxed SIRT1 transgenic mouse, referred to as SIRT1^{STOP} (FIG. 1B). SIRT1 was cloned downstream of a constitutive CAAGS promoter and a transcriptional loxP-STOP-loxP cassette, then integrated into an FRT site at the collagen locus of ES cells (ColA1) using Flp recombinase (15) (FIG. 1C and D). SIRT1^{STOP} transgenic mice were generated from the ES cells and crossed to a C57BL/6 Villin-Cre (Vil-Cre) strain (16), which generated progeny with the STOP cassette excised specifically in gut villi (SIRT1^{ΔSTOP}). Vil-Cre; SIRT1^{ΔSTOP} mice expressed SIRT1 at levels in gut epithelial cells similar to those under CR conditions (FIG. 1E). The morphology of villi oveexpressing SIRT1 appeared otherwise normal (Figure F). The Vil-Cre SIRT1^{ΔSTOP} mice were bred to APC^{min/-} mice to generate a triple transgenic SIRT1^{ΔSTOP}; Vil-Cre; APC^{min/+} strain.

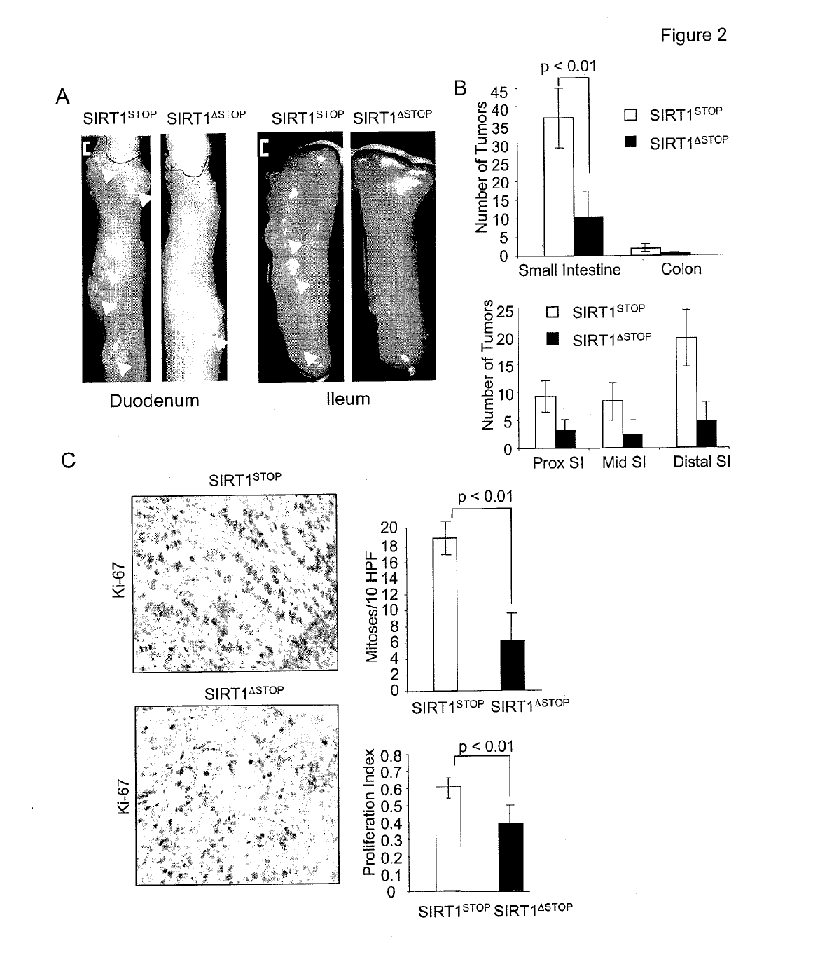

The APC^{min/+} mice showed the typical increase in morbidity compared to wild type controls at 16 weeks of age, as evidenced by overt anemia and loss of body weight, whereas the SIRT1 transgenic APC^{min/+} mice displayed none of these symptoms (FIG. S1). Examination of the gut lining at four months of age showed that the SIRT1 transgenic mice had significantly smaller tumors and fewer of them, both in the duodenum and ileum (FIG. 2A). Quantification of the tumor burden showed a 3- to 4-fold reduction in the number and size of adenomas within the small intestine and colon of the SIRT1^{ΔSTOP} transgenic (FIG. 2B). Ki-67 is a granular component of the nucleolus that is expressed exclusively in proliferating cells and is used as a prognostic marker in human neoplasias. The SIRT1^{ΔSTOP} mice had a significant reduction in the numbers of mitoses (per high-power field) and Ki-67 staining, demonstrating there was less cellular proliferation in the tumors of the transgenic mice (FIG. 2C). Thus, overexpression of SIRT1 in the gut at similar levels to those induced by CR is sufficient to mimic the effect of CR on tumorigenesis in the APC^{min/-} mouse.

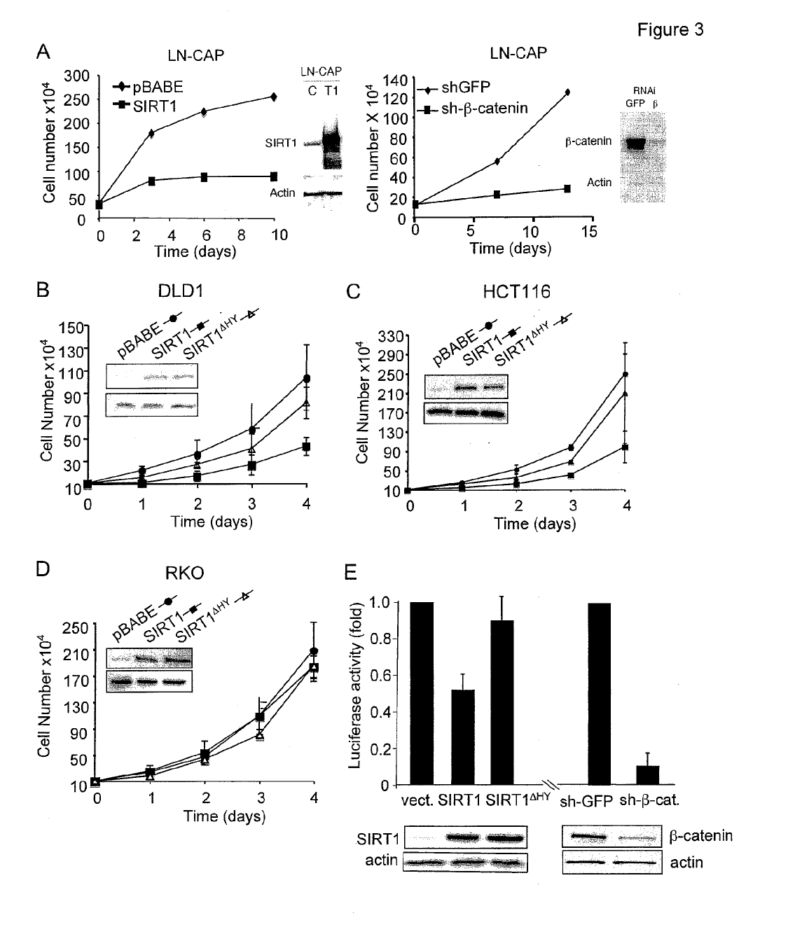

To gain insights into the mechanisms by which SIRT1 reduces cellular proliferation, we examined the effect of SIRT1 on the growth rate of several well characterized cancer cell lines. LN-CAP is a human colon cancer cell line driven by aberrant β-catenin activity. The proliferation of LN-CAP cells was greatly reduced by overexpression of SIRT1 and the effect was similar to knocking down β-catenin itself (FIG. 3A). This result suggested that SIRT1 might reduce cellular proliferation by suppressing β-catenin activity. To further explore this possibility, we expressed SIRT1 and a catalytically inactive SIRT1 mutant (SIRT1^{ΔHY}) in a variety of other cell lines whose growth is driven by constitutively active β-catenin (LN-CAP, HCT116, and DLD1). A cell line in which β-catenin is inactive (RKO) served as a negative control. Increased SIRT1 expression greatly reduced proliferation in all three of the cell lines with constitutively active β-catenin but not in the β-catenin-inactive cell line (FIG. 3A-D). The SIRT1^{ΔHY} catalytic mutant had no significant effect on cellular proliferation in any of the cell lines (FIG. 3B-D). Thus, SIRT1 suppresses β-catenin-driven proliferation and its catalytic activity is required for the effect.

To further understand the mechanism by which SIRT1 suppresses β-catenin-driven proliferation, we engineered the DLD1 human colon cancer cell line to contain a stably integrated reporter with β-catenin response elements (Super8XTopflash-Luciferase^{PEST}). Knockdown of β-catenin dramatically reduced reporter activity, demonstrating that reporter activity was driven by endogenous β-catenin (FIG. 3E). Overexpression of SIRT1 reduced reporter activity by ˜2-fold, whereas the SIRT1^{ΔHY} catalytic mutant had no effect (FIG. 3E), suggesting that the anti-proliferative effects of SIRT1 are mediated by its ability to suppress the transcriptional activity of endogenous β-catenin and that this requires SIRT1 deacetylase activity.

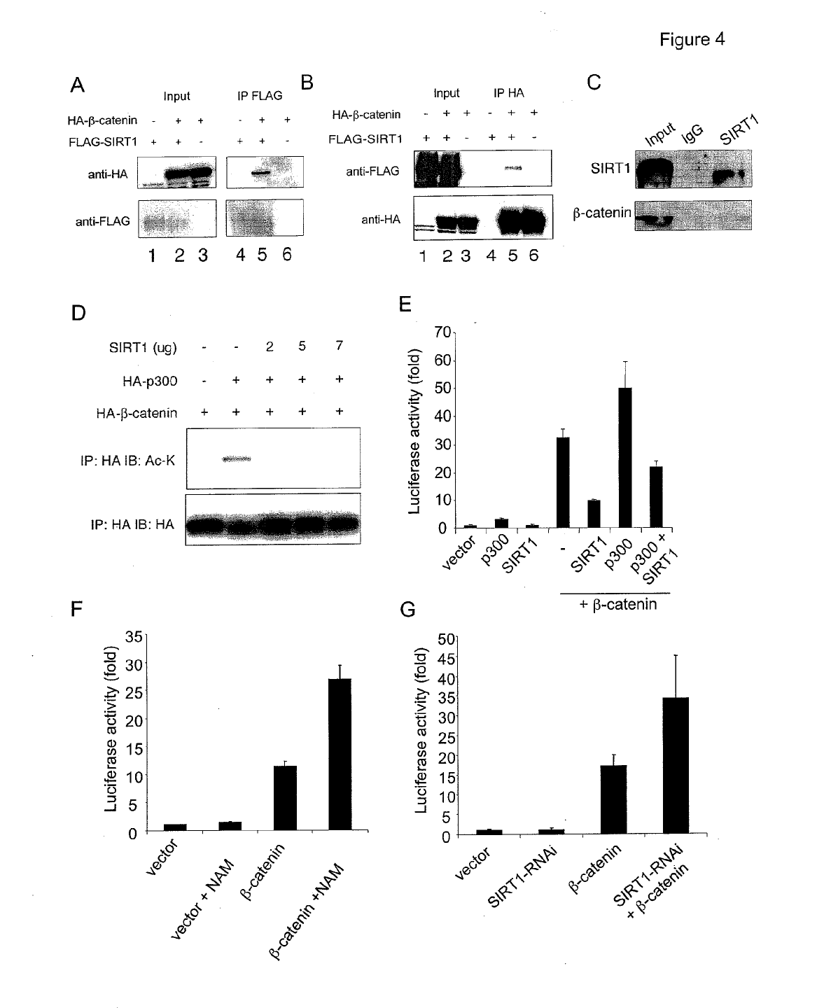

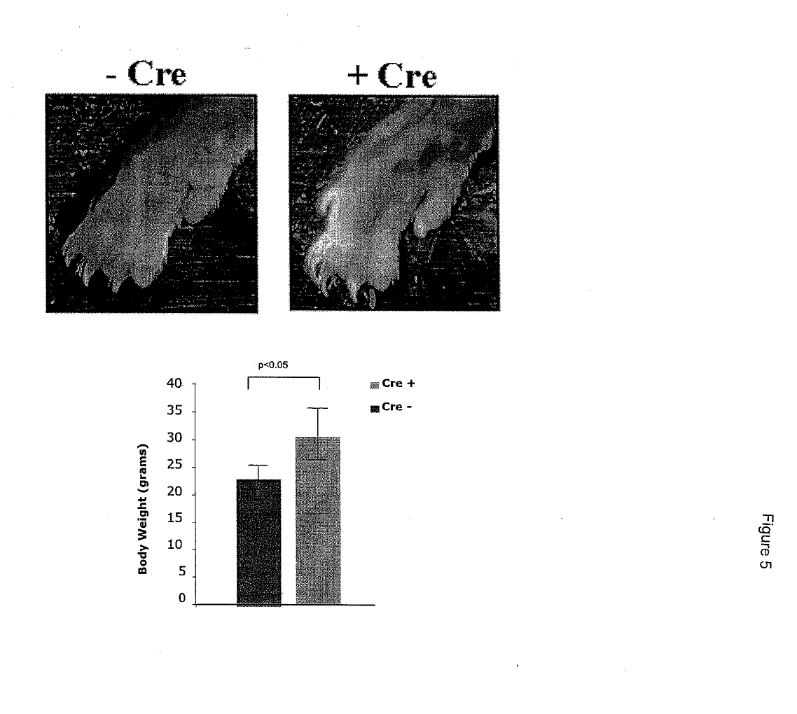

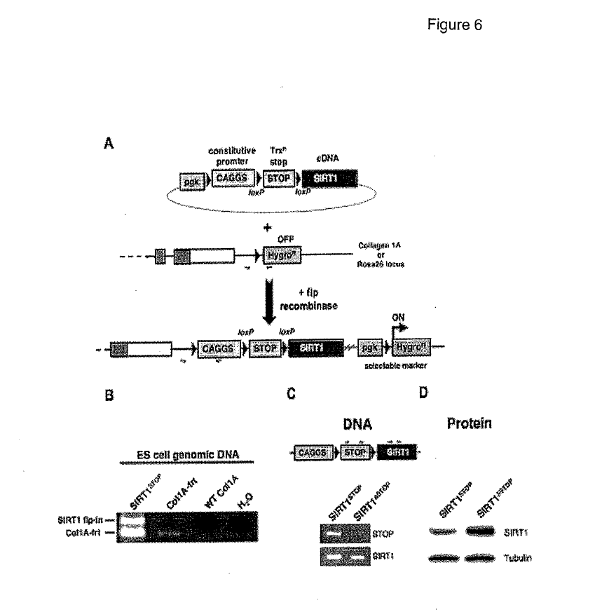

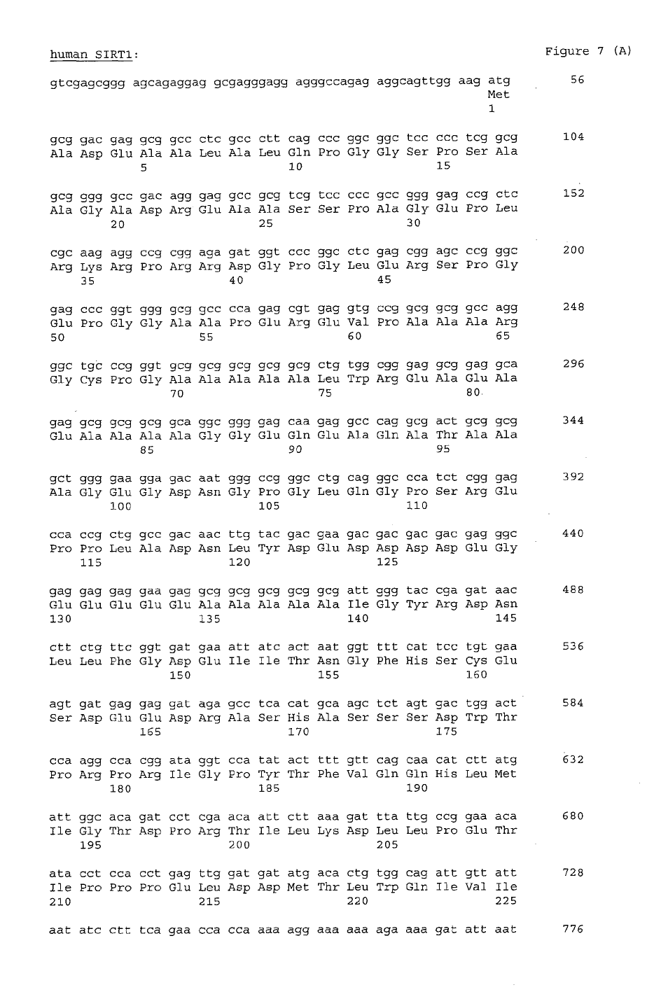

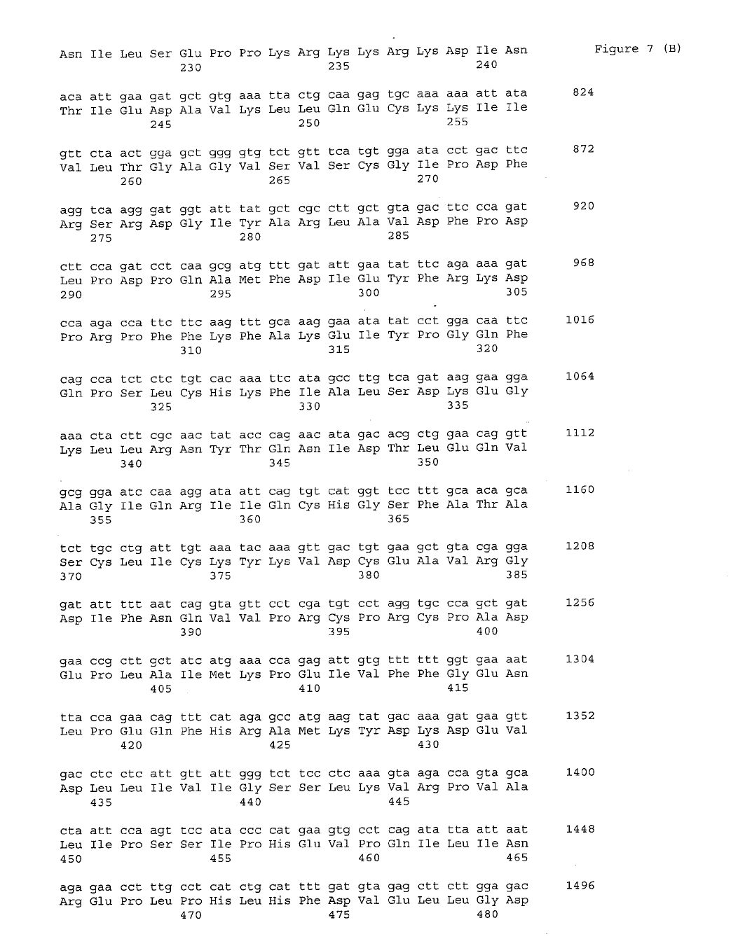

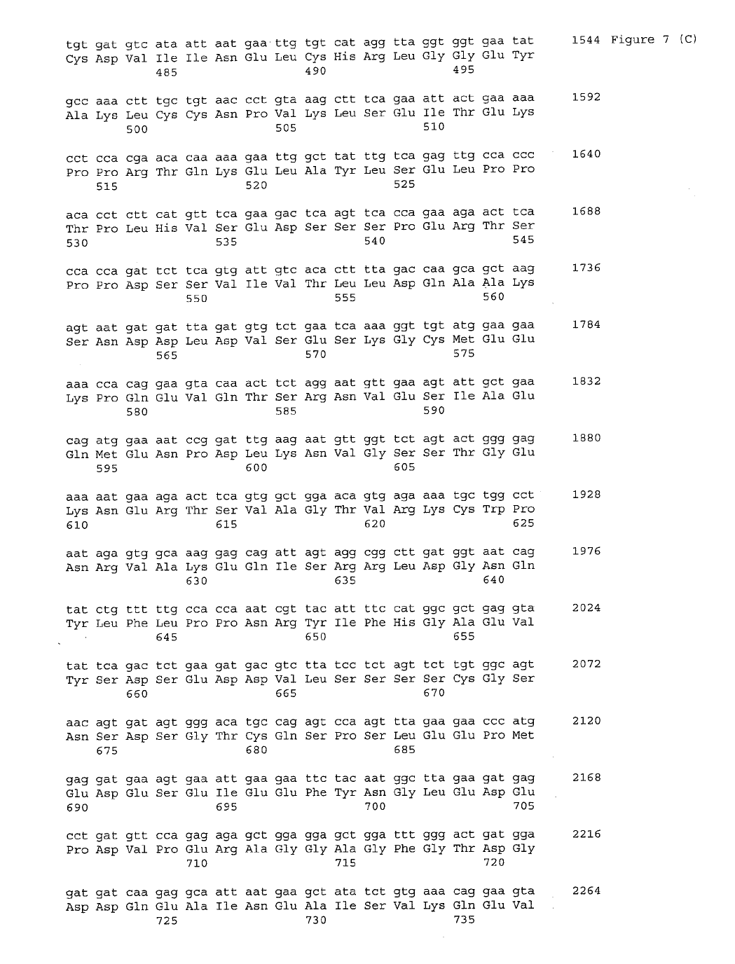

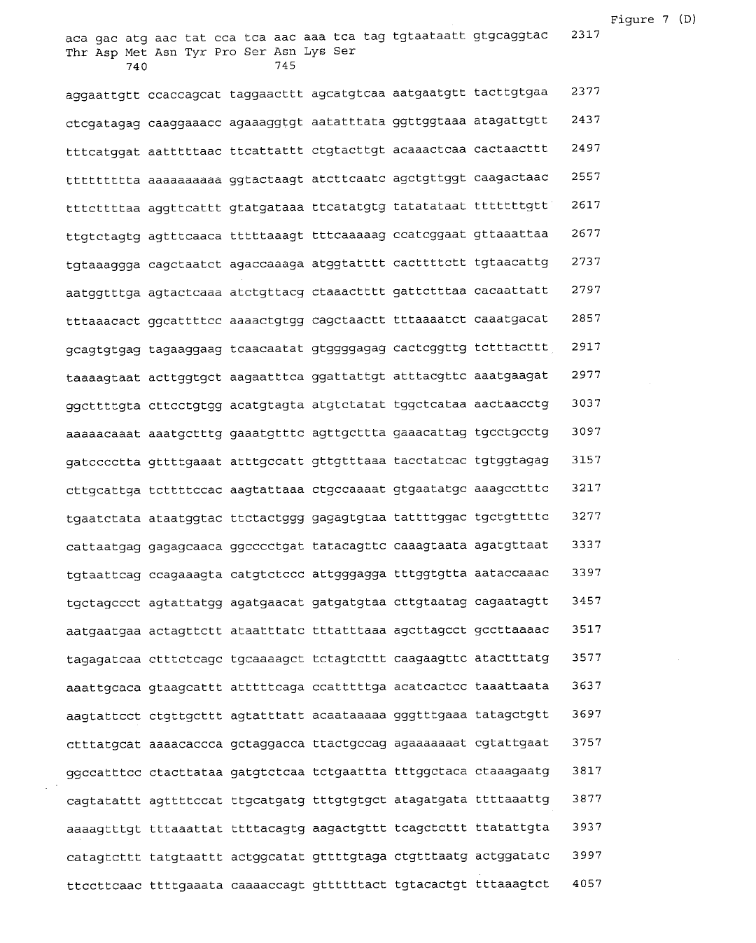

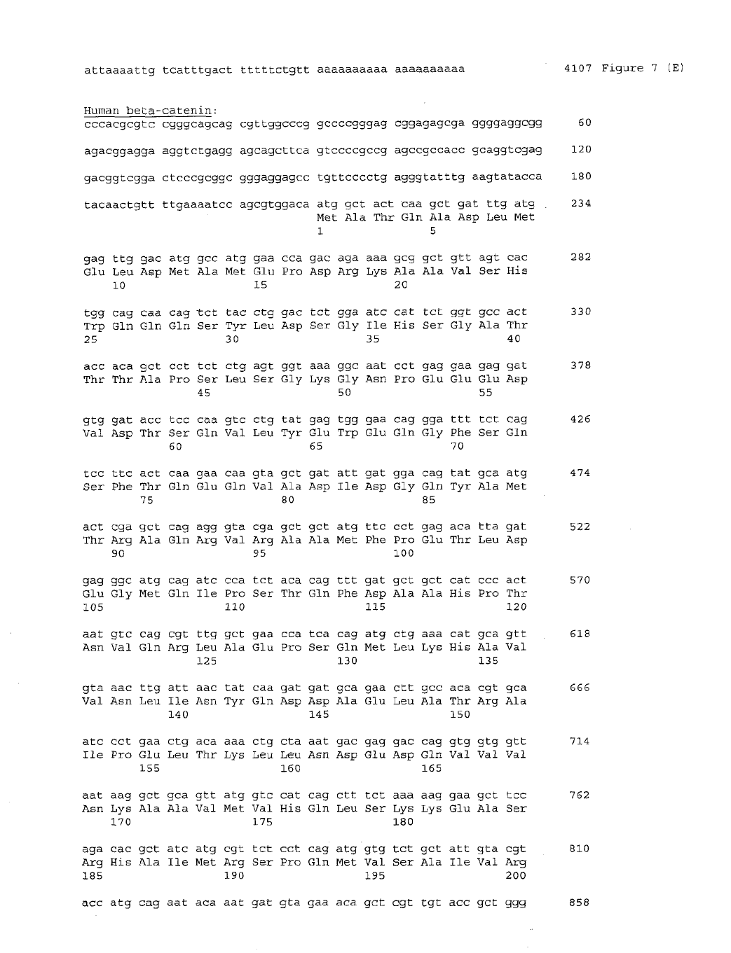

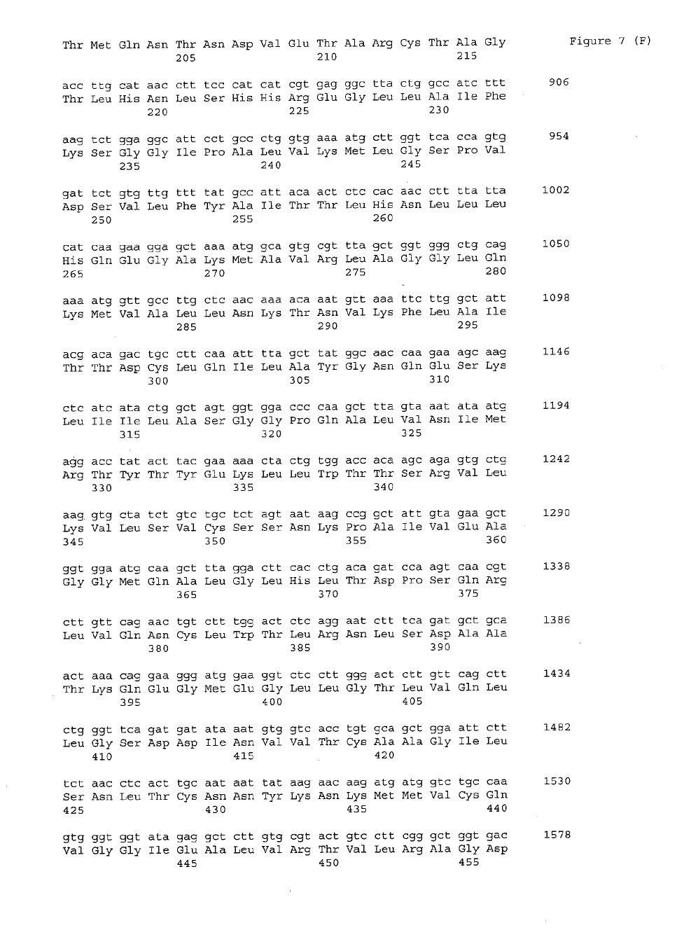

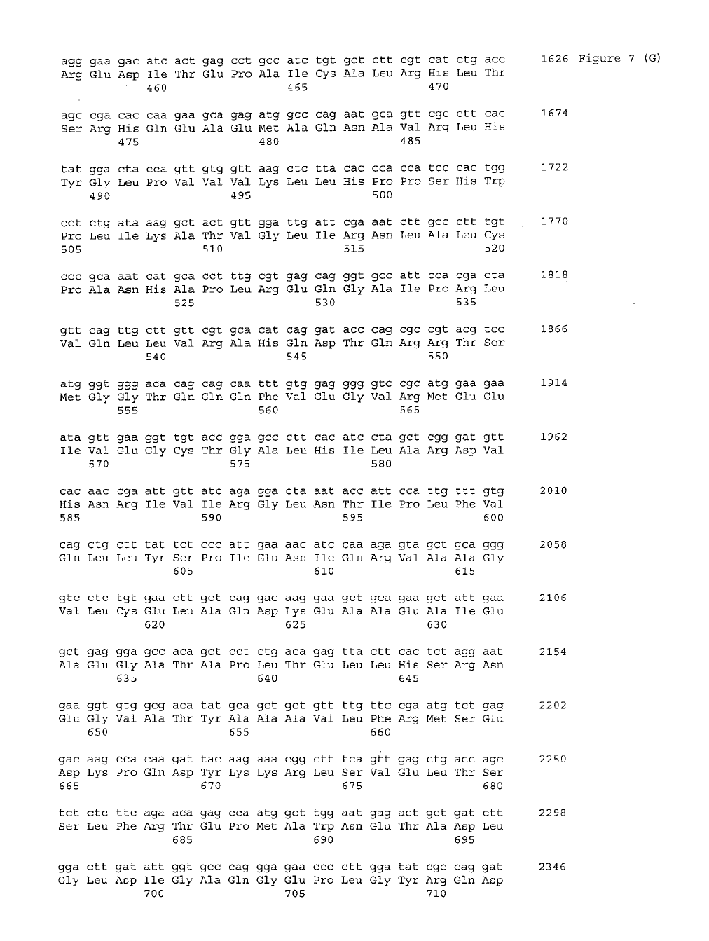

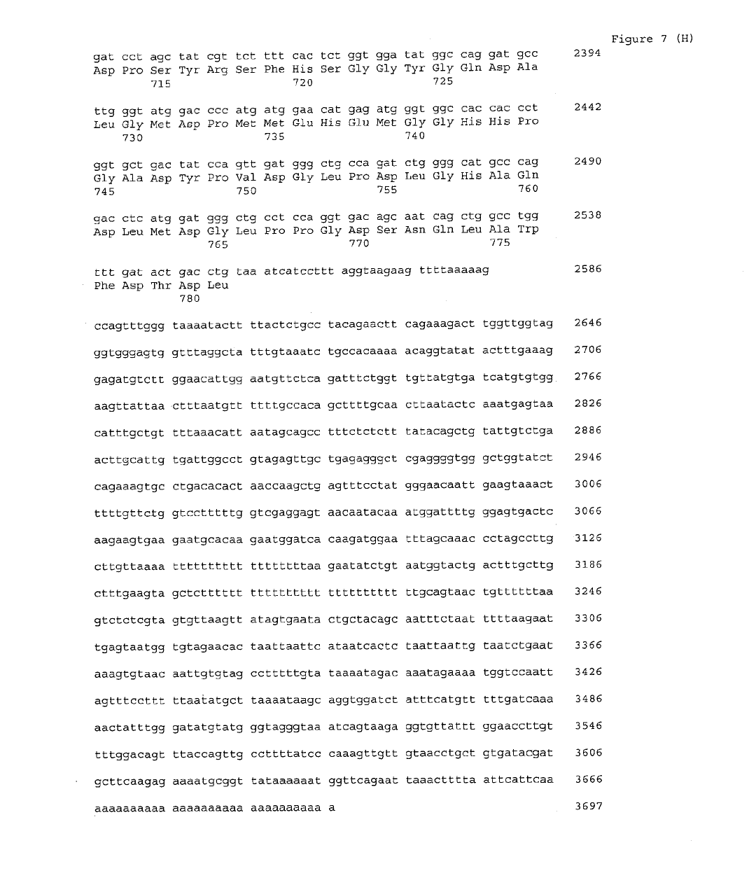

Recent studies have shown that β-catenin exists in an acetylated form that has a higher affinity for TCF/LEF-1 and hence a greater ability to activate target genes (17-19). These observations led us to speculate that SIRT1 may be exerting its inhibitory effects by de acetylating β-catenin. To explore this possibility, we first tested whether SIRT1 and β-catenin physically interact. HEK293T cells were transfected with a mutant form of β-catenin that constitutively localizes to the nucleus (S33Y-β-catenin) (20). In these cells, SIRT1 co-immunoprecipitated with β-catenin (FIG. 4A) and vice versa (FIG. 4B). Co-immunoprecipitation of endogenous SIRT1 and β-catenin also revealed a direct interaction between the two proteins (FIG. 4C).

Next, we tested whether SIRT1 is capable of deacetylating β-catenin. 293T cells were transfected with S33Y-β-catenin, the acetyltransferase p300, and increasing amounts of SIRT1. Acetylated β-catenin was detected in cells co-transfected with p300 and SIRT1 expression almost completely abolished this signal (top panel), suggesting that SIRT1 can deacetylate β-catenin (FIG. 4D).

To test the effect of SIRT1-mediated deacetylation of β-catenin we utilized a “pTOPFLASH” β-catenin reporter (21). As previously shown (18), expression of β-catenin increased luciferase activity and co-transfection with a p300 expression plasmid further increased luciferase activity (FIG. 4E). SIRT1 significantly reduced luciferase activity when co-transfected with either β-catenin or β-catenin and p300 (FIG. 4E). Conversely, treating cells with the SIRT1 inhibitor nicotinamide (NAM) (22), or knocking down SIRT1 with a retroviral siRNA vector, increased luciferase reporter activity (FIG. 4F, 4G). Together, these data indicate that SIRT1 deacetylates β-catenin, thereby reducing its ability to act as a transactivator.

In this study we have shown that SIRT1 inhibits cellular proliferation of β-catenin-positive cell lines and suppresses tumorigenesis in the APCmin/+ mouse model. We also show that SIRT1 deacetylates β-catenin and reduces its ability to transactivate gene transcription, possibly explaining the in vivo effects of SIRT1 in this model. The decreased number and size of tumors in the SIRT1 transgenic and reduced proliferation within the tumors, suggest that SIRT1 can suppress tumor growth even after tumors have initiated. Based on these data, we propose that SIRT1 upregulation may be the basis for tumor suppressive effects of CR in the APC^{min/+} model, and that activation of SIRT1 may prove a useful avenue for treating human cancers that are driven by mutations in the wnt/β-catenin signaling pathway.

### Material and Methods

### Rodents

A Cre-inducible SIRT1 expression construct was generated in which a loxP flanked transcriptional STOP element was inserted between a CAGGS promoter and the SIRT1 cDNA. This construct was targeted into the mouse Collagen 1A locus using flp recombinase-mediated genomic integration as described previously (1). ES cells carrying a single copy of the SIRT1-STOP construct were identified by resistance to the antibiotic marker hygromycin and Southern blotting. PCR primers and construct maps are available upon request. Two clones were injected into blastocysts and both generated pups, ˜90% of which displayed germ-line transmission. Tumor bearing mice that were analyzed had been backcrossed at least four generations into C57/BL6. APC^{min/+} and Villin-Cre transgenic mice strains were obtained in the C57/BL6 background from Jackson Labs (Bar Harbor, Me.). SirT1^{STOP} animals were backcrossed two generations into C57BL/6 mice before crossing first to APC^{min/+} animals to generate SirT1^{STOP}; APC^{min/+} double transgen These animals were bred to Villin-Cre transgenic mice to generate a cohort of SirT1^{ΔSTOP}; Vil-Cre; APC^{min/+} animals. Animals were maintained at Harvard Medical School and experiments were approved by the Animal Care Committee of Harvard Medical School.

Male Fischer-344 (F344) rats were bred and reared in a vivarium at the Gerontology Research Center (GRC, Baltimore, Md.). From weaning (2 Wks), the rats were housed individually in standard plastic cages with beta chip wood bedding. Control animals were fed a NIH-31 standard diet ad libitum (AL). At 1 month of age the calorie restricted (CR) animals were provided a vitamin and mineral fortified version of the same diet at a level of 40% less food (by weight) than AL rats consumed during the previous week. Filtered and acidified water was available ad libitum for both groups. The vivarium was maintained at a temperature of 25° C. with relative humidity at 50% on a 12/12-hour light/dark cycle (lights on at 6:00 a.m.) All animals were 6 months of age and sacrificed between 9:00 and 11:00 a.m. The intestine was quickly removed and thoroughly flushed with ice cold PBS and placed into liquid nitrogen then stored at −80 ° C. until processed for Western blotting using standard procedures.

### Pathology, Histopathology and Immunohistochemical Analysis

For gross tumor analysis, the entire intestine was excised immediately after sacrifice, opened lengthwise and washed with cold phosphate-buffered saline (PBS) while pinned down a solid support. Adenomas larger than 0.5 mm from the proximal (10 cm distal to the pylorus), distal (10 cm proximal to the cecum), and middle (˜50% of total intestinal length) small intestine as well as the colon were scored. Intestines were prepared using the Swiss roll method by rotating them around a glass pipette tip. Tissues were fixed and embedded in paraffin using standard histology protocol. Precise tumor size was scored microscopically on hematoxylin/eosin stained of mouse intestines using a microscope with an eyepiece micrometer. Immunohistochemical analysis was performed with rabbit anti-SIRT1 antibody (Upstate Biotechnology, cat #07-131), rabbit anti-β catenin (abcam #2982) and rat anti-mouse Ki-67 (Dako).

### Plasmids

pcDNA3-FLAG-SIRT1, pBABE-Puro-hSir2(SIRT1), pBABE-Puro-SIRT1^{ΔHY}, pcDNA-HA-S33Y-β-catenin and pBABE-Puro-S33Y-β-catenin have been described before. SIRT1 RNAi plasmids were constructed by cloning the sequences into the pSUPER.retro plasmid (oligoEngine, Seattle, Wash.). One TOPFLASH plasmid was purchased from Upstate Biotechnology (Lake Placid, N.Y.) while the second was generated by cloning the tandem TCF binding sites and TA-promoter from SUPER8xTOPFLASH (kind gift of Randall Moon) into the Luciferase-PEST plasmid pGL4.15 (Promega, Madison, Wis.).

### Cell Transfections and Infections

293T, LN-CAP, DLD1, HCT116 and RKO cells were maintained in the recommended tissue culture media (American Type Culture Collection (ATCC), Manassas, Va.) and grown in a humidified incubator containing CO₂ (5% v/v) at 37° C. For over-expression experiments, plasmids were transfected by the Fugene 6 method (Roche). For stable cell line generation, DLD1 cells were selected in hygromycin for two weeks and single colonies were picked and expanded. For retroviral production, 293T cells were transfected with the overexpression or RNAi plasmids simultaneously with packaging plasmids gag-pol and VSV-G or pCL-ampho. The media containing the progeny virus released for the 293T cells was collected and used to infect the cells for 3-6 hours in the presence of 8 μg/ml polybrene (Sigma Aldrich, St. Louis, Mo.). The medium was changed and cells were incubated for an additional 24-48 hour incubation. They were selected with puromycin (Sigma Aldrich) for 24-48 hours and then trypsinized and seeded for experiments.

### Protein Extraction and Immunoprecipitation

Cell extracts analyzed directly by Western blotting were prepared by cell lysis in 1× SDS loading buffer followed by boiling and Western analysis. Cell extracts for immunoprecipitation were prepared by resuspending phosphate-buffered saline-washed cell pellets in 1 ml of Nonidet P-40 (NP-40) extraction buffer (50 mM Tris-HCl [pH 8.0], 150 mM NaCl, and 1% Nonidet P-40) supplemented with EDTA-free protease inhibitor cocktail tablets (Roche) with 10 mM Nicotinamide (NAA) and 5 uM Trichostatin-A (TSA). Following incubation on ice for 30 minutes, nonextractable material was removed by centrifugation at 17,000 g for 10 min at 4 ° C., and the cleared supernatants were employed for immunoprecipitation. Lysates were immunoprecipitated (2 hr), washed 4 times with NP-40 buffer and were proceeded by Western blotting.

### Proliferation Assays

DLD1, HCT116 and RKO cell lines infected with the appropriate construct were seeded in 24 well plates at a density of 10,000 cells. Cells were trypsinized and analyzed by Coulter Counting at given time points.

### Luciferase Assay

Luciferase assay was done as instructed by the dual luciferase reporter assay system or firefly luciferase assay system (Promega, Madison, Wis.).

### Western Blotting and RNA Analysis

For expression studies, animal intestines were flushed with cold PBS and either homogenized whole or scraped to enrich for enterocytes. Protein extracts were prepared by dounce homogenization in standard lysis buffer, subjected to SDS-PAGE and transferred onto PVDF membranes. Membranes were immunoblotted using rabbit polyclonal antibody to SIRT1 (Upstate Biotechnology, cat #07-131) and rabbit polyclonal antibody to β-actin (Abcam cat #8226). Densitometric analysis was performed on scanned images of blots using ImageJ software (NIH Image analysis website http://rsb.info.nih.gov/ij/).

### REFERENCES AND NOTES

1. D. Sinclair. Mech Ageing Dev. 126, 987 (2005).
2. L. Guarente, F. Picard. Cell 120, 473 (2005).
3. C. S. Lim. Med Hypotheses (2006).
4. F. Yeung et al., EMBO J 23, 2369 (2004).
5. J. Ford et al., Cancer Res 65, 10457 (2005).
6. H. Y. Cohen et al., Science 305, 390 (2004).
7. H. Vaziri et al., Cell 107, 149 (2001).
8. K. Pruitt et al., PLoS Genet 2, e40 (2006).
9. W. Y. Chen et al., Cell 123, 437 (2005).
10. M. Fu et al., Mol Cell Biol 26, 8122 (2006).
11. K. F. Chua et al., Cell Metab 2, 67 (2005).
12. C. Y. Logan, R. Nusse. Annu Rev Cell Dev Biol 20, 781 (2004).
13. A. R. Moser et al., Eur J Cancer 31A, 1061 (1995).
14. A. C. Patel et al., J Nutr 134, 3394S (2004).
15. C. Beard et al., Genesis 44, 23 (2006).
16. F. el Marjou et al., Genesis 39, 186 (2004).
17. D. Wolf et al., J Biol Chem 277, 25562 (2002).
18. L. Levy et al., Mol Cell Biol 24, 3404 (2004).
19. A. Hecht et al., EMBO J 19, 1839 (2000).
20. I. Simcha et al., J. Cell Biol. 141,1433 (1988).
21. V. Korinek et al., Science 275, 1784 (1997).
22. K. J. Bitterman et al., J Biol Chem 277, 45099. (2002).

### Example 2

### Cellular Localization of SIRT1 and β-Catenin

Experimental results have indicated the existence of a positive trend between high SIRT1 and membraneous beta-catenin, and inverse correlation between high SIRT1 and nuclear beta-catenin. When SIRT1 is overexpressed in colon cancer cells, beta catenin is more in the membrane and perinuclear area.

### Example 3

### Overexpression of SIRT1

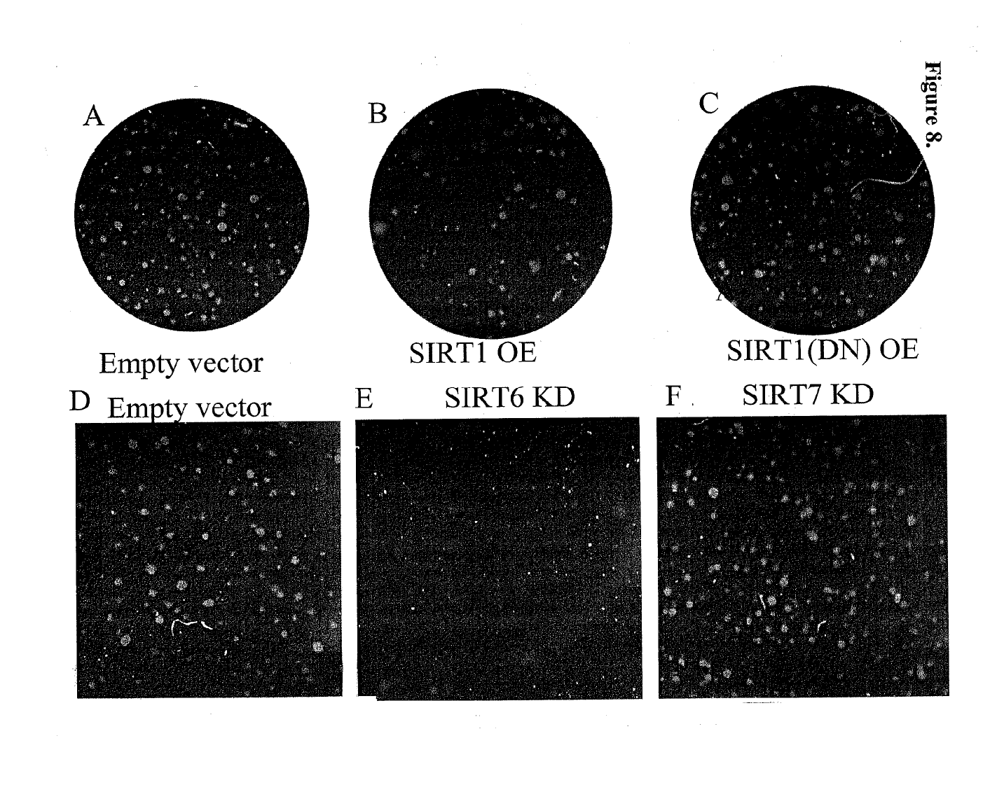

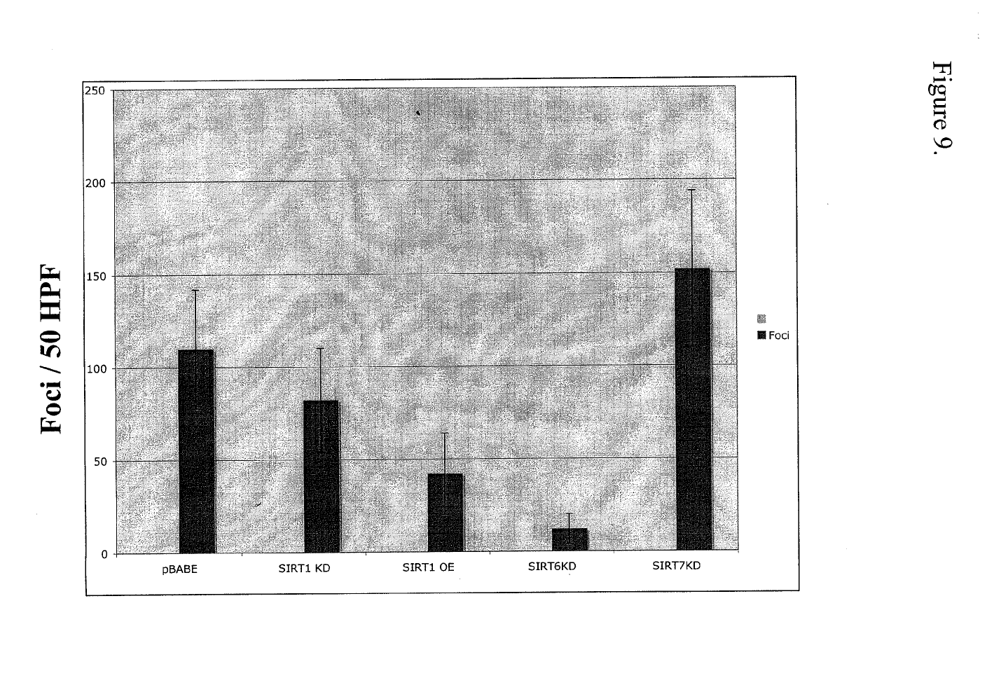

Demonstrated herein is the interaction between SIRT1 and β-catenin, wherein SIRT1 suppresses β-catenin-associated gene transcription and reduces cellular proliferation. In addition to increased cell proliferation, cancerous cells are marked by abnormal cell-cell and cell-matrix adhesions. It is shown here that SIRT1 also increases cellular adhesive capacity. As shown in FIG. 8, overexpression of SIRT1 (“SIRT1 OE”, shown in FIG. 8B) reduces colony formation in soft agar; however, overexpression of a dominant-negative, catalytically-inactive SIRT1 (“SIRT1(DN) OE”, shown in FIG. 8C) results in no reduction in colony formation, as compared to an empty vector control (“Empty vector”). FIG. 9 quantitatively demonstrates that overexpression of SIRT1 (“SIRT1 OE”) reduces foci formation as compared to an empty vector control (“pBABE”).

### Example 4

### Modulation of Stem Cell Dynamics with Sirtuin Agents

The Wnt pathway, particularly beta-catenin, is essential for stem/progenitor cell function, expansion, and maintenance in normal tissue during embryogenesis, tissue regeneration, and adult cell renewal (See, Gregorieff and Clevers (2005) Genes & Dev. 19: 877-890). Sirtuin-activating agents are useful in prolonging pluripotency in stem cells, and preventing aberrant stem cell proliferation, such as in certain cancers. Sirtuin inhibitors are therefore useful in activating Wnt signaling and therefore inducing tissue regeneration by differentiating stem/progenitor cells. For example, Sirtuin inhibitors are useful to regenerate intestinal epithelial tissue damaged by inflammatory bowel disease, islet cells damaged or destroyed in diabetic subjects, and hepatic tissue in subjects affected by alcohol abuse or hepatitis viruses.

All publications, patents, patent applications and GenBank Accession numbers mentioned herein are hereby incorporated by reference in their entirety as if each individual publication or patent was specifically and individually indicated to be incorporated by reference. In case of conflict, the present application, including any definitions herein, will control.

### Equivalents

Those skilled in the art will recognize, or be able to ascertain using no more than routine experimentation, many equivalents to the specific embodiments of the invention described herein. Such equivalents are intended to be encompassed by the following claims.

1. A method for treating a disease associated with a dysregulated activation of β-catenin activity in a subject, comprising administering to a subject in need thereof a therapeutically effective amount of an agent that increases the level or activity of a sirtuin.

2. The method of claim 1, wherein the sirtuin is SIRT1.

3. The method of claim 2, wherein the agent is a compound having a formula selected from the group of formulas set forth herein.

4. The method of claim 1, wherein the disease is cancer.

5. The method of claim 4, wherein the disease is an age-related cancer.

6. The method of claim 5, wherein the cancer is colon cancer.

7. The method of claim 6, wherein the method reduces the number and size of adenomas within the small intestine and colon.

8. The method of claim 6, wherein the method reduces colon tumor morbidity.

9. The method of claim 6, wherein the agent acts on the intestinal tract of the subject.

10. The method of claim 9, wherein the agent is contacted with the intestinal tract of the subject.

11. The method of claim 9, wherein the agent is a heterologous nucleic acid encoding SIRT1 that is expressed in the intestinal tract of the subject.

12. The method of claim 1, wherein the disease is a wound and the method improves wound healing.

13. The method of claim 1, wherein the disease is selected from the group consisting of fibromatosis, Dupuytren's disease, polycystic kidney disease (ADPKD), Hailey-Hailey disease and Sjorgen's disease.

14. The method of claim 1, comprising determining whether β-catenin has a dysregulated activation.

15. The method of claim 14, comprising obtaining a biological sample from the subject and determining the level of activation of β-catenin in the subject.

16. The method of claim 4, comprising obtaining a sample of a tumor of the subject, determining the level of activation of β-catenin therein, and if the level of activation of β-catenin is elevated relative to a control level, administering to the subject an agent that increases the level or activity of a sirtuin.

17-21. (canceled)

22. A method for suppressing β-catenin-driven proliferation of a cell, comprising providing a cell whose proliferation is driven by β-catenin; and contacting the cell with an agent that increases sirtuin level or activity.

23. The method of claim 22, comprising: providing a cell; determining whether the proliferation of the cell is driven by β-catenin; and contacting a cell whose proliferation is driven by β-catenin with an agent that increases sirtuin level or activity.

24. (canceled)

25. A method for identifying an agent that modulates the interaction between a sirtuin and a β-catenin protein, comprising contacting a composition comprising a sirtuin or homolog thereof sufficient for interacting with a β-catenin protein and a β-catenin protein or homolog thereof sufficient to interact with a sirtuin with a test agent under conditions in which the sirtuin or homolog thereof and the β-catenin or homolog thereof interact in the absence of the test agent, wherein a difference in the level of interaction between the sirtuin or homolog thereof and the β-catenin or homolog thereof indicates that the test agent modulates the interaction.

26. The method of claim 25, wherein the method is for identifying an agent that inhibits the interaction between a sirtuin and a β-catenin protein and a lower level of interaction between the sirtuin or homolog thereof and the β-catenin or homolog thereof indicates that the test agent inhibits the interaction.

27-40. (canceled)


SEQUENCE LISTING (4 sequences)

SEQ ID NO: 1 - DNA, 4107 bp, Homo sapiens
gtcgagcgggagcagaggaggcgagggaggagggccagagaggcagttggaagatggcggacgaggcggccctcgcccttcagcccggcggctccccctcggcggcgggggccgacagggaggccgcgtcgtcccccgccggggagccgctccgcaagaggccgcggagagatggtcccggcctcgagcggagcccgggcgagcccggtggggcggccccagagcgtgaggtgccggcggcggccaggggctgcccgggtgcggcggcggcggcgctgtggcgggaggcggaggcagaggcggcggcggcaggcggggagcaagaggcccaggcgactgcggcggctggggaaggagacaatgggccgggcctgcagggcccatctcgggagccaccgctggccgacaacttgtacgacgaagacgacgacgacgagggcgaggaggaggaagaggcggcggcggcggcgattgggtaccgagataaccttctgttcggtgatgaaattatcactaatggttttcattcctgtgaaagtgatgaggaggatagagcctcacatgcaagctctagtgactggactccaaggccacggataggtccatatacttttgttcagcaacatcttatgattggcacagatcctcgaacaattcttaaagatttattgccggaaacaatacctccacctgagttggatgatatgacactgtggcagattgttattaatatcctttcagaaccaccaaaaaggaaaaaaagaaaagatattaatacaattgaagatgctgtgaaattactgcaagagtgcaaaaaaattatagttctaactggagctggggtgtctgtttcatgtggaatacctgacttcaggtcaagggatggtatttatgctcgccttgctgtagacttcccagatcttccagatcctcaagcgatgtttgatattgaatatttcagaaaagatccaagaccattcttcaagtttgcaaaggaaatatatcctggacaattccagccatctctctgtcacaaattcatagccttgtcagataaggaaggaaaactacttcgcaactatacccagaacatagacacgctggaacaggttgcgggaatccaaaggataattcagtgtcatggttcctttgcaacagcatcttgcctgatttgtaaatacaaagttgactgtgaagctgtacgaggagatatttttaatcaggtagttcctcgatgtcctaggtgcccagctgatgaaccgcttgctatcatgaaaccagagattgtgttttttggtgaaaatttaccagaacagtttcatagagccatgaagtatgacaaagatgaagttgacctcctcattgttattgggtcttccctcaaagtaagaccagtagcactaattccaagttccataccccatgaagtgcctcagatattaattaatagagaacctttgcctcatctgcattttgatgtagagcttcttggagactgtgatgtcataattaatgaattgtgtcataggttaggtggtgaatatgccaaactttgctgtaaccctgtaaagctttcagaaattactgaaaaacctccacgaacacaaaaagaattggcttatttgtcagagttgccacccacacctcttcatgtttcagaagactcaagttcaccagaaagaacttcaccaccagattcttcagtgattgtcacacttttagaccaagcagctaagagtaatgatgatttagatgtgtctgaatcaaaaggttgtatggaagaaaaaccacaggaagtacaaacttctaggaatgttgaaagtattgctgaacagatggaaaatccggatttgaagaatgttggttctagtactggggagaaaaatgaaagaacttcagtggctggaacagtgagaaaatgctggcctaatagagtggcaaaggagcagattagtaggcggcttgatggtaatcagtatctgtttttgccaccaaatcgttacattttccatggcgctgaggtatattcagactctgaagatgacgtcttatcctctagttcttgtggcagtaacagtgatagtgggacatgccagagtccaagtttagaagaacccatggaggatgaaagtgaaattgaagaattctacaatggcttagaagatgagcctgatgttccagagagagctggaggagctggatttgggactgatggagatgatcaagaggcaattaatgaagctatatctgtgaaacaggaagtaacagacatgaactatccatcaaacaaatcatagtgtaataattgtgcaggtacaggaattgttccaccagcattaggaactttagcatgtcaaaatgaatgtttacttgtgaactcgatagagcaaggaaaccagaaaggtgtaatatttataggttggtaaaatagattgtttttcatggataatttttaacttcattatttctgtacttgtacaaactcaacactaacttttttttttttaaaaaaaaaaaggtactaagtatcttcaatcagctgttggtcaagactaactttcttttaaaggttcatttgtatgataaattcatatgtgtatatataattttttttgttttgtctagtgagtttcaacatttttaaagttttcaaaaagccatcggaatgttaaattaatgtaaagggacagctaatctagaccaaagaatggtattttcacttttctttgtaacattgaatggtttgaagtactcaaaatctgttacgctaaacttttgattctttaacacaattatttttaaacactggcattttccaaaactgtggcagctaactttttaaaatctcaaatgacatgcagtgtgagtagaaggaagtcaacaatatgtggggagagcactcggttgtctttacttttaaaagtaatacttggtgctaagaatttcaggattattgtatttacgttcaaatgaagatggcttttgtacttcctgtggacatgtagtaatgtctatattggctcataaaactaacctgaaaaacaaataaatgctttggaaatgtttcagttgctttagaaacattagtgcctgcctggatccccttagttttgaaatatttgccattgttgtttaaatacctatcactgtggtagagcttgcattgatcttttccacaagtattaaactgccaaaatgtgaatatgcaaagcctttctgaatctataataatggtacttctactggggagagtgtaatattttggactgctgttttccattaatgaggagagcaacaggcccctgattatacagttccaaagtaataagatgttaattgtaattcagccagaaagtacatgtctcccattgggaggatttggtgttaaataccaaactgctagccctagtattatggagatgaacatgatgatgtaacttgtaatagcagaatagttaatgaatgaaactagttcttataatttatctttatttaaaagcttagcctgccttaaaactagagatcaactttctcagctgcaaaagcttctagtctttcaagaagttcatactttatgaaattgcacagtaagcatttatttttcagaccatttttgaacatcactcctaaattaataaagtattcctctgttgctttagtatttattacaataaaaagggtttgaaatatagctgttctttatgcataaaacacccagctaggaccattactgccagagaaaaaaatcgtattgaatggccatttccctacttataagatgtctcaatctgaatttatttggctacactaaagaatgcagtatatttagttttccatttgcatgatgtttgtgtgctatagatgatattttaaattgaaaagtttgttttaaattatttttacagtgaagactgttttcagctctttttatattgtacatagtcttttatgtaatttactggcatatgttttgtagactgtttaatgactggatatcttccttcaacttttgaaatacaaaaccagtgttttttacttgtacactgttttaaagtctattaaaattgtcatttgacttttttctgttaaaaaaaaaaaaaaaaaaaa

SEQ ID NO: 2 - protein, 747 aa, Homo sapiens
MADEAALALQPGGSPSAAGADREAASSPAGEPLRKRPRRDGPGLERSPGEPGGAAPEREVPAAARGCPGAAAAALWREAEAEAAAAGGEQEAQATAAAGEGDNGPGLQGPSREPPLADNLYDEDDDDEGEEEEEAAAAAIGYRDNLLFGDEIITNGFHSCESDEEDRASHASSSDWTPRPRIGPYTFVQQHLMIGTDPRTILKDLLPETIPPPELDDMTLWQIVINILSEPPKRKKRKDINTIEDAVKLLQECKKIIVLTGAGVSVSCGIPDFRSRDGIYARLAVDFPDLPDPQAMFDIEYFRKDPRPFFKFAKEIYPGQFQPSLCHKFIALSDKEGKLLRNYTQNIDTLEQVAGIQRIIQCHGSFATASCLICKYKVDCEAVRGDIFNQVVPRCPRCPADEPLAIMKPEIVFFGENLPEQFHRAMKYDKDEVDLLIVIGSSLKVRPVALIPSSIPHEVPQILINREPLPHLHFDVELLGDCDVIINELCHRLGGEYAKLCCNPVKLSEITEKPPRTQKELAYLSELPPTPLHVSEDSSSPERTSPPDSSVIVTLLDQAAKSNDDLDVSESKGCMEEKPQEVQTSRNVESIAEQMENPDLKNVGSSTGEKNERTSVAGTVRKCWPNRVAKEQISRRLDGNQYLFLPPNRYIFHGAEVYSDSEDDVLSSSSCGSNSDSGTCQSPSLEEPMEDESEIEEFYNGLEDEPDVPERAGGAGFGTDGDDQEAINEAISVKQEVTDMNYPSNKS

SEQ ID NO: 3 - DNA, 3697 bp, Homo sapiens
cccacgcgtccgggcagcagcgttggcccggccccgggagcggagagcgaggggaggcggagacggaggaaggtctgaggagcagcttcagtccccgccgagccgccaccgcaggtcgaggacggtcggactcccgcggcgggaggagcctgttcccctgagggtatttgaagtataccatacaactgttttgaaaatccagcgtggacaatggctactcaagctgatttgatggagttggacatggccatggaaccagacagaaaagcggctgttagtcactggcagcaacagtcttacctggactctggaatccattctggtgccactaccacagctccttctctgagtggtaaaggcaatcctgaggaagaggatgtggatacctcccaagtcctgtatgagtgggaacagggattttctcagtccttcactcaagaacaagtagctgatattgatggacagtatgcaatgactcgagctcagagggtacgagctgctatgttccctgagacattagatgagggcatgcagatcccatctacacagtttgatgctgctcatcccactaatgtccagcgtttggctgaaccatcacagatgctgaaacatgcagttgtaaacttgattaactatcaagatgatgcagaacttgccacacgtgcaatccctgaactgacaaaactgctaaatgacgaggaccaggtggtggttaataaggctgcagttatggtccatcagctttctaaaaaggaagcttccagacacgctatcatgcgttctcctcagatggtgtctgctattgtacgtaccatgcagaatacaaatgatgtagaaacagctcgttgtaccgctgggaccttgcataacctttcccatcatcgtgagggcttactggccatctttaagtctggaggcattcctgccctggtgaaaatgcttggttcaccagtggattctgtgttgttttatgccattacaactctccacaaccttttattacatcaagaaggagctaaaatggcagtgcgtttagctggtgggctgcagaaaatggttgccttgctcaacaaaacaaatgttaaattcttggctattacgacagactgccttcaaattttagcttatggcaaccaagaaagcaagctcatcatactggctagtggtggaccccaagctttagtaaatataatgaggacctatacttacgaaaaactactgtggaccacaagcagagtgctgaaggtgctatctgtctgctctagtaataagccggctattgtagaagctggtggaatgcaagctttaggacttcacctgacagatccaagtcaacgtcttgttcagaactgtctttggactctcaggaatctttcagatgctgcaactaaacaggaagggatggaaggtctccttgggactcttgttcagcttctgggttcagatgatataaatgtggtcacctgtgcagctggaattctttctaacctcacttgcaataattataagaacaagatgatggtctgccaagtgggtggtatagaggctcttgtgcgtactgtccttcgggctggtgacagggaagacatcactgagcctgccatctgtgctcttcgtcatctgaccagccgacaccaagaagcagagatggcccagaatgcagttcgccttcactatggactaccagttgtggttaagctcttacacccaccatcccactggcctctgataaaggctactgttggattgattcgaaatcttgccctttgtcccgcaaatcatgcacctttgcgtgagcagggtgccattccacgactagttcagttgcttgttcgtgcacatcaggatacccagcgccgtacgtccatgggtgggacacagcagcaatttgtggagggggtccgcatggaagaaatagttgaaggttgtaccggagcccttcacatcctagctcgggatgttcacaaccgaattgttatcagaggactaaataccattccattgtttgtgcagctgctttattctcccattgaaaacatccaaagagtagctgcaggggtcctctgtgaacttgctcaggacaaggaagctgcagaagctattgaagctgagggagccacagctcctctgacagagttacttcactctaggaatgaaggtgtggcgacatatgcagctgctgttttgttccgaatgtctgaggacaagccacaagattacaagaaacggctttcagttgagctgaccagctctctcttcagaacagagccaatggcttggaatgagactgctgatcttggacttgatattggtgcccagggagaaccccttggatatcgccaggatgatcctagctatcgttcttttcactctggtggatatggccaggatgccttgggtatggaccccatgatggaacatgagatgggtggccaccaccctggtgctgactatccagttgatgggctgccagatctggggcatgcccaggacctcatggatgggctgcctccaggtgacagcaatcagctggcctggtttgatactgacctgtaaatcatcctttaggtaagaagttttaaaaagccagtttgggtaaaatacttttactctgcctacagaacttcagaaagacttggttggtagggtgggagtggtttaggctatttgtaaatctgccacaaaaacaggtatatactttgaaaggagatgtcttggaacattggaatgttctcagatttctggttgttatgtgatcatgtgtggaagttattaactttaatgttttttgccacagcttttgcaacttaatactcaaatgagtaacatttgctgttttaaacattaatagcagcctttctctctttatacagctgtattgtctgaacttgcattgtgattggcctgtagagttgctgagagggctcgaggggtgggctggtatctcagaaagtgcctgacacactaaccaagctgagtttcctatgggaacaattgaagtaaactttttgttctggtcctttttggtcgaggagtaacaatacaaatggattttgggagtgactcaagaagtgaagaatgcacaagaatggatcacaagatggaatttagcaaaccctagccttgcttgttaaaattttttttttttttttttaagaatatctgtaatggtactgactttgcttgctttgaagtagctcttttttttttttttttttttttttttttgcagtaactgttttttaagtctctcgtagtgttaagttatagtgaatactgctacagcaatttctaatttttaagaattgagtaatggtgtagaacactaattaattcataatcactctaattaattgtaatctgaataaagtgtaacaattgtgtagcctttttgtataaaatagacaaatagaaaatggtccaattagtttcctttttaatatgcttaaaataagcaggtggatctatttcatgtttttgatcaaaaactatttgggatatgtatgggtagggtaaatcagtaagaggtgttatttggaaccttgttttggacagtttaccagttgccttttatcccaaagttgttgtaacctgctgtgatacgatgcttcaagagaaaatgcggttataaaaaatggttcagaattaaacttttaattcattcaaaaaaaaaaaaaaaaaaaaaaaaaaaaaaaaa

SEQ ID NO: 4 - protein, 781 aa, Homo sapiens
MATQADLMELDMAMEPDRKAAVSHWQQQSYLDSGIHSGATTTAPSLSGKGNPEEEDVDTSQVLYEWEQGFSQSFTQEQVADIDGQYAMTRAQRVRAAMFPETLDEGMQIPSTQFDAAHPTNVQRLAEPSQMLKHAVVNLINYQDDAELATRAIPELTKLLNDEDQVVVNKAAVMVHQLSKKEASRHAIMRSPQMVSAIVRTMQNTNDVETARCTAGTLHNLSHHREGLLAIFKSGGIPALVKMLGSPVDSVLFYAITTLHNLLLHQEGAKMAVRLAGGLQKMVALLNKTNVKFLAITTDCLQILAYGNQESKLIILASGGPQALVNIMRTYTYEKLLWTTSRVLKVLSVCSSNKPAIVEAGGMQALGLHLTDPSQRLVQNCLWTLRNLSDAATKQEGMEGLLGTLVQLLGSDDINVVTCAAGILSNLTCNNYKNKMMVCQVGGIEALVRTVLRAGDREDITEPAICALRHLTSRHQEAEMAQNAVRLHYGLPVVVKLLHPPSHWPLIKATVGLIRNLALCPANHAPLREQGAIPRLVQLLVRAHQDTQRRTSMGGTQQQFVEGVRMEEIVEGCTGALHILARDVHNRIVIRGLNTIPLFVQLLYSPIENIQRVAAGVLCELAQDKEAAEAIEAEGATAPLTELLHSRNEGVATYAAAVLFRMSEDKPQDYKKRLSVELTSSLFRTEPMAWNETADLGLDIGAQGEPLGYRQDDPSYRSFHSGGYGQDALGMDPMMEHEMGGHHPGADYPVDGLPDLGHAQDLMDGLPPGDSNQLAWFDTDL